(19) United States
(12) Patent Application Publication
(10) Pub. No.: US 2023/0074301 A1
(43) Pub. Date: Mar. 9, 2023

(54) COMPOSITIONS COMPRISING PIG STOMACH MUCINS AND USES THEREOF

(71) Applicants: Adeyemi ADESOKAN, Lausanne (CH); Sara Vidal LÓPEZ, Spiez (CH); Yong MIAO, Lausanne (CH); Gnubiotics Sciences SA, Epalinges (CH)

(72) Inventors: Adeyemi Adesokan, Lausanne (CH); Sara Vidal López, Spiez (CH); Yong Miao, Lausanne (CH)

(21) Appl. No.: 17/796,628

(22) PCT Filed: Feb. 1, 2021
(86) PCT No.: PCT/IB2021/000050

§ 371 Date: Jul. 29, 2022

Related U.S. Application Data
(60) Provisional application No. 62/968,039, filed on Jan. 30, 2020.

(51) Int. Cl. C07K 14/47; A61K 35/38; A61P 1/00; A61P 29/00; A61P 35/00; A61K 45/06; A61K 38/17; A23L 33/00; A23L 33/18; A23K 20/147

(52) U.S. Cl. CPC C07K 14/4727; A61K 35/38; A61P 1/00; A61P 29/00; A61P 35/00; A61K 45/06; A61K 38/1735; A23L 33/40; A23L 33/18; A23K 20/147; A23V 2002/00

(57) ABSTRACT

Disclosed herein are methods of making compositions having high glycoprotein content and low free glycan content from porcine gastric mucus. Also disclosed are compositions having high glycoprotein content and low free glycan content as well as methods of using the same, including for the treatment of cancer, inflammatory bowel disease, and acute colitis.

### RELATED APPLICATION(S)

This application claims the benefit of U.S. Provisional Application No. 62/968,039, filed on Jan. 30, 2020, the contents of which are hereby incorporated by reference in their entirety.

### BACKGROUND OF THE INVENTION

It has been recently recognized that the dense microbial community (microbiota) present in the mammalian, and in particular human, intestine shortly after birth and throughout the life has a profound effect on health and physiology.

One major factor shaping the composition and physiology of the microbiota is the influx of glycans into the intestine, mostly from diet and host mucosal secretions. Humans consume dozens of different plant- and animal-derived dietary glycans, most of which cannot be degraded by enzymes encoded in the human genome. Microbial fermentation transforms these indigestible glycans into short chain fatty acids which serve as nutrients for colonocytes and other gut epithelial cells (i.e., intestinal epithelial cells). Gut microorganisms therefore play a pivotal symbiotic role in helping mammals (e.g., humans, dogs, cats, and livestock) access calories from otherwise indigestible nutrients and each type of microorganisms prefer different glycans. Therefore, a selective consumption of nutrients can influence which microbial groups proliferate and persist in the gastrointestinal tract. Dietary glycans have been considered as being a possible non-invasive strategy of directly influencing the balance of bacterial species in the gut (Koropathkin et al., 2012, Nat Rev Microbial. 10(5): 323-35).

Gut microbes also play an important role in the regulation of host metabolism and low-grade inflammation. Abnormalities in microbiota composition and activity (called dysbiosis) have been implicated in the emergence of the metabolic syndrome, which include diseases such as obesity, type 2 diabetes and cardiovascular diseases.

### SUMMARY OF THE INVENTION

The present disclosure is directed to methods and compositions with glycoproteins obtained from porcine gastric mucus (“pig slime”). Surprisingly, it has been found that compositions from pig slime can be obtained with minimal processing that have very high glycoprotein content and very low free glycan content. Such compositions are useful as prebiotics for enhancing gut microbiota, for example to administer to infants or toddlers.

Some aspects of the present disclosure are directed to a composition comprising a mixture of glycoproteins obtained from mucins of the outer mucus layer of pig stomach, wherein: a) the composition is obtained without subjecting the mucins to conditions or reagents that release oligosaccharides from glycoproteins and glycopeptides; b) glycoprotein content of the composition is greater than about 70% (w/w); and c) the free glycan content of the composition is less than 1% (w/w). In some embodiments, the oligosaccharide content of the composition is greater than or equal to about 35% (w/w). In some embodiments, the composition has a salt content of less than about 2%. In some embodiments, the composition is a powder and has a glycoprotein content of greater than 75% by weight. In some embodiments, the composition has a free glycan content of less than 0.1% by weight.

In some embodiments, the composition is a nutritional or dietary composition, nutritional or dietary premix, or infant formula. In some embodiments, the composition is an animal feed or animal feed supplement. In some embodiments, the composition is a liquid or slurry for administration to an infant (e.g., newborn).

Some aspects of the present disclosure are directed to a method of manufacturing a composition comprising a mixture of glycopeptides, comprising the following steps a)-g): a) providing a composition comprising mucins from the outer mucus layer of pig stomach or a partially purified fraction thereof and water; b) adjusting the pH of the composition to 3.0 to 3.5 with the addition of an acid and incubating the solution to hydrolyze the composition; c) isolating an aqueous phase from the composition; d) defatting the isolated aqueous phase; e) precipitating and isolating a composition comprising glycopeptides from the defatted aqueous phase; f) dewatering the isolated composition; and g) drying the dewatered composition to obtain a composition comprising a mixture of glycopeptides; wherein the composition comprising a mixture of glycopeptides has an glycopeptide content of greater than or equal to about 70% (w/w) and has a free glycan content of less than 1% (w/w).

In some embodiments, the composition of step a) has been homogenized. In some embodiments, the composition of step a) comprises about a 1:1 ratio of pig stomach outer mucus layer to water. In some embodiments, the pH is adjusted in step b) with HCl. In some embodiments, the composition is incubated in step b) at a pH of 3.0 to 3.5 for 2-4 hours at 45° C. In some embodiments, step b) further comprises adding 1 part of an aqueous solution having a pH of 3.0 to 3.5 to 2-3 parts of the composition after incubation. In some embodiments, the aqueous phase is isolated in step c) by a process comprising centrifugation followed by removal of the aqueous phase. In some embodiments, the aqueous phase obtained in step c) is filtered to remove insoluble material prior to step d). In some embodiments, the isolated aqueous phase is defatted in step d) by the addition of about 5% v/w heptane or hexane followed by incubation for 6-18 hours and removal of the heptane or hexane phase. In some embodiments, the defatted aqueous phase is filtered to remove insoluble material prior to step e). In some embodiments, the defatted aqueous phase is concentrated to ½ to ¼ of the initial volume prior to step e). In some embodiments, the composition is precipitated in step e) with ethanol or acetone at about 4° C. In some embodiments, the composition is isolated in step e) by filtration or centrifugation after precipitation. In some embodiments, the composition is dewatered in step f) with ethanol. In some embodiments, drying the dewatered composition of step g) comprises freeze drying or rotary evaporation. In some embodiments, the composition of step b) comprises pepsin. In some embodiments, the composition of step a) has not been subject to conditions or reagents that release oligosaccharides from glycoproteins and glycopeptides.

Some aspects of the present disclosure are directed to a composition comprising a mixture of glycoproteins obtained by the methods disclosed herein.

Some aspects of the present disclosure are directed to a method of treating, preventing, or reducing the severity of a pathogenic microorganism infection of the gut of a subject comprising orally administering to the subject a composition disclosed herein or a composition made by a method disclosed herein. In some embodiments, the pathogenic microorganism is selected from Escherichia coli, Helicobacter pylori, Streptococcus spp., Toxoplasma gondii, Plasmodium falciparum, influenza virus, rotavirus, and respirovirus. In some embodiments, the pathogenic microorganism is Escherichia coli.

Some aspects of the present disclosure are directed to a method of increasing the growth of commensal bacteria in the gut of a subject comprising orally administering to the subject a composition disclosed herein or a composition made by a method disclosed herein. In some embodiments, the commensal bacteria comprise Lactobacillus acidophilus, Lactobacillus reuteri, Akkermansia mucimphila, Bacteroides thetaiotaomicron, Bifidobacterium breve, and/or Bifidobacterium infantis.

Some aspects of the present disclosure are directed to a method of reducing the fat mass of a subject comprising orally administering to the subject a composition disclosed herein or a composition made by a method disclosed herein.

Some aspects of the present disclosure are directed to a method of treating, preventing, or reducing inflammation in a subject comprising orally administering to the subject a composition disclosed herein or a composition made by a method disclosed herein. In some embodiments, administration of the composition reduces a level of calprotectin in the blood stream or stool of the subject.

Some aspects of the present disclosure are directed to a method of increasing production of short chain fatty acid (SCFA) in the gut of a subject comprising orally administering to the subject a composition disclosed herein or a composition made by a method disclosed herein. In some embodiments, the pH in the gut of the subject is decreased.

Some aspects of the present disclosure are directed to a method of improving gut barrier integrity in the gut of a subject comprising orally administering to the subject a composition disclosed herein or a composition made by a method disclosed herein.

Some aspects of the present invention are related to a method of assisting the development of beneficial gut microbiota in an infant comprising orally administering to the infant a composition disclosed herein or a composition manufactured by a method disclosed herein. In some embodiments, the infant is a newborn. In some embodiments, the newborn was delivered by caesarean section (C-section). In some embodiments, the beneficial gut microbiota comprises one or more of Lactobacillus acidophilus, Lactobacillus reuteri, Akkermansia muciniphila, Bacteroides thetaiotaomicron, Bifidobacterium breve, and/or Bifidobacterium infantis. In some embodiments, the beneficial gut microbiota includes a decreased level of a pathogenic microorganism. In some embodiments, the pathogenic microorganism is one or more of Escherichia coli, Helicobacter pylori, Streptococcus spp., Toxoplasma gondii, Plasmodium falciparum, influenza virus, rotavirus, and/or respirovirus.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, Bacteroides (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, available on the World Wide Web at ncbi.nlm.nih.gov/omim/and in Online Mendelian Inheritance in Animals (OMIA), a database of genes, inherited disorders and traits in animal species (other than human and mouse), at omia.angis.org.au/contact.shtml.

Some aspects of the present disclosure are related to a method of treating a cancer in a subject in need thereof, comprising administering to the subject a composition described herein or a composition made by a method described herein. In some embodiments, the cancer is a solid tumor cancer. In some embodiments, the cancer is an immunotherapy responsive cancer. In some embodiments, the cancer is a checkpoint inhibitor responsive cancer. In some embodiments, the checkpoint inhibitor is an anti-PD-1 antibody, an anti-CTLA-4 antibody, an anti-PD-L1 antibody, or an anti-PD-L2. In some embodiments, the checkpoint inhibitor is nivolumab, pembrolizumab, atezolizumab, durvalumab, pidilizumab, PDR001, BMS-936559, avelumab, or SHR-1210. In some embodiments, the checkpoint inhibitor is nivolumab.

In some embodiments, the cancer is adrenal cancer, biliary cancer, bladder cancer, brain cancer, breast cancer, cervical cancer, colon cancer, colorectal cancer, rectum cancer, endometrial cancer, esophageal cancer, head or neck cancer, kidney cancer, liver cancer, non-small cell lung cancer, lung cancer, lymphoma, melanoma, meninges cancer, non-melanoma skin cancer, ovarian cancer, pancreatic cancer, prostate cancer, sarcoma, small intestine cancer, or stomach cancer. In some embodiments, the cancer is melanoma or colorectal cancer. In some embodiments, the subject is further administered the checkpoint inhibitor. In some embodiments, the subject is periodically administered the composition starting at least 1 week, at least 2 weeks, or at least 1 month prior to administration of the checkpoint inhibitor. In some embodiments, the subject is periodically administered the composition at the same time as the checkpoint inhibitor or starting about 1 week, 2 weeks, or at least 1 month after administration of the checkpoint inhibitor. In some embodiments, periodic administration of the composition comprises administration at least once per day, at least once every other day, or at least once every three days. In some embodiments, the composition is orally administered.

In some embodiments, administration of the composition increases microbiota diversity in the gut of the subject. In some embodiments, administration of the composition increases infiltration of immune cells into tumors. In some embodiments, the immune cells are selected from CD4+ T-cells; IFN-γ+, Foxp3+ T-cells; CD8+ T-cells; dendritic cells; plasmacytoid dendritic cells; B cells; macrophages; and natural-killer cells. In some embodiments, administration of the composition reduces systemic inflammation in the subject. In some embodiments, a systemic level of one or more inflammatory cytokines is reduced. In some embodiments, the inflammatory cytokines are selected from Eotaxin, G-CSF, GM-CSF, IFN-γ, IL-1α, IL-1β, IL-2, IL-3, IL-4, IL-5, IL-6, IL-9, IL-10, IL-12, IL-13, IL-17A, KC, MCP-1 (MCAF), MIP-1α, MIP-1β, RANTES, or TNF-α. In some embodiments, administration of the composition increases cancer cell death.

In some embodiments, administration of the composition increases production of short chain fatty acid (SCFA) in the gut of the subject. In some embodiments, administration of the composition reduces the side effects of an anti-cancer therapy administered to the subject. In some embodiments, the subject is human.

Some aspects of the present disclosure are related to a method of treating inflammation in a subject in need thereof, comprising administering to the subject a composition described herein or a composition made by a method described herein. In some embodiments, the subject has inflammatory bowel disease or acute colitis. In some embodiments, the composition is administered at least once per day, at least once every other day, or at least once every three days. In some embodiments, the composition is orally administered. In some embodiments, administration of the composition increases microbiota diversity in the gut of the subject. In some embodiments, a systemic level of one or more inflammatory cytokines is reduced. In some embodiments, the inflammatory cytokines are selected from Eotaxin, G-CSF, GM-CSF, IFN-γ, IL-1α, IL-1β, IL-2, IL-3, IL-4, IL-5, IL-6, IL-9, IL-10, IL-12, IL-13, IL-17A, KC, MCP-1 (MCAF), MIP-1α, MIP-1β, RANTES, or TNF-α. In some embodiments, administration of the composition increases production of short chain fatty acid (SCFA) in the gut of the subject. In some embodiments, the subject is human.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a diagram of a process used to obtain a composition of the claimed invention from the outer mucus layer of pig stomach (i.e., pig slime, pig stomach slime, slime).

FIG. 2 shows pig slime after homogenization with an electric homogenizer for 1-2 minutes.

FIG. 3 shows homogenized pig slime after hydrolysis with acid.

FIG. 4 shows hydrolyzed slime after centrifugation separated into fat (top), aqueous (middle), and sediment phases.

FIG. 5 shows the fat phase after isolation from the aqueous and sediment phases.

FIG. 6 shows the sediment phase after isolation from the aqueous and sediment phases.

FIG. 7 shows further filtration of the aqueous phase. Solid material removed by filtration is shown in the left panel. Aqueous filtrate is shown in the right panel.

FIG. 8 shows equal amounts of the fat (left vial), aqueous (middle vial), and solid (right vial) after centrifugation.

FIG. 9 shows 5% heptane extraction of the aqueous phase. Left panel is a close-up of the heptane/aqueous interface. Right panel shows the extraction without stirring (left bottle) and with stirring (right panel).

FIG. 10 shows separated heptane phase and aqueous phase. Left panel shows separation of heptane and aqueous phase via a separatory funnel. Right panel shows the defatted aqueous phase (left bottle) and heptane phase (right bottle).

FIG. 11 shows defatted aqueous phase after centrifugation to separate remaining fat (left panel), followed by filtration to remove fat layer (right panel).

FIG. 12 (left panel) shows defatted aqueous phase before (left bottle) and after (right bottle) concentration in a rotary evaporator to one-third original volume. FIG. 12 (right panel) shows the concentrated aqueous phase after overnight incubation at 4° C.

FIG. 13 shows concentrated aqueous phase after overnight incubation at 4° C. (left panel) and after addition of ethanol (right panel).

FIG. 14 shows ethanol precipitate. Top left panel shows precipitate in bottle. Top right panel shows a pellet of precipitate after centrifugation. Bottom panel shows isolated precipitate.

FIG. 15 shows a second ethanol precipitation of the supernatant after removal of precipitated glycoproteins. The second precipitation was performed at a higher ethanol concentration to remove any non-precipitated glycoproteins. Top left panel shows supernatant prior to second precipitation. Top right panel shows supernatant in 90% ethanol (left bottle). Bottom left panel shows supernatant after second precipitation and centrifugation. Bottom right panel shows precipitate after second ethanol precipitation and isolation.

FIG. 16 shows dewatering of precipitate. Top left panel shows addition of pure ethanol to precipitate. Top right panel shows isolated precipitate after removal of pure ethanol. Bottom panel shows turbid ethanol supernatant.

FIG. 17 shows mixing of the precipitation with 80% ethanol and stirred overnight at 4° C. (top left panel); pellet from the second step (top right panel); and transparent supernatant from the second step (bottom panel).

FIG. 18 shows dissolution of the precipitate in ultrapure water (top left panel); dissolved sample with the insoluble dark fragments (top right panel); dissolved product with the dark fragments isolated (bottom panel).

FIG. 19 shows dried insoluble dark fragment (top left panel); freeze dried final product (top right panel); milled powder of the final product (bottom panel).

FIG. 20 shows a graph (left side) and an enlarged region of the same graph (right side) to show detail for Lactobacillus acidophilus growth in minimal media (no glucose), minimal media with glucose, and with GBX102.

FIG. 21 shows a graph (left side) and an enlarged region of the same graph (right side) to show detail for Lactobacillus reuteri growth in minimal media (no glucose), minimal media with glucose, and with GBX102.

FIG. 22 shows a graph (left side) and an enlarged region of the same graph (right side) to show detail for Akkermansia muciniphila growth in minimal media (no glucose), minimal media with glucose, and with GBX102.

FIG. 23 shows a graph (left side) and an enlarged region of the same graph (right side) to show detail for Bacteroides thetaiotaomicron growth in minimal media (no glucose), minimal media with glucose, and with GBX102.

FIG. 24 shows a graph (left side) and an enlarged region of the same graph (right side) to show detail for Bifidobacterium breve growth in minimal media (no glucose), minimal media with glucose, and with GBX102 at various concentrations and time points. Percentage value concentrations shown are in reference to 15 mg/ml. Thus, 100% equals 15 mg/ml, 50% equals 7.5 mg/ml, etc.

FIG. 25 shows a graph (left side) and an enlarged region of the same graph (right side) to show detail for Lactobacillus acidophilus growth in minimal media (no glucose), minimal media with glucose, and with GBX102 at various concentrations and time points. Percentage value concentrations shown are in reference to 15 mg/ml. Thus, 100% equals 15 mg/ml, 50% equals 7.5 mg/ml, etc.

FIG. 26 shows a graph (left side) and an enlarged region of the same graph (right side) to show detail for Lactobacillus reuteri growth in minimal media (no glucose), minimal media with glucose, and with GBX102 at various concentrations and time points. Percentage value concentrations shown are in reference to 15 mg/ml. Thus, 100% equals 15 mg/ml, 50% equals 7.5 mg/ml, etc.

FIG. 27 shows a graph for Akkermansia muciniphila growth in minimal media (no glucose), minimal media with glucose, and with GBX102 at various concentrations and time points. Percentage value concentrations shown are in reference to 15 mg/ml. Thus, 100% equals 15 mg/ml, 50% equals 7.5 mg/ml, etc.

FIG. 28 shows a graph for Bacteroides thetaiotaomicron growth in minimal media (no glucose), minimal media with glucose, and with GBX102 at various concentrations and time points. Percentage value concentrations shown are in reference to 15 mg/ml. Thus, 100% equals 15 mg/ml, 50% equals 7.5 mg/ml, etc.

FIG. 29 shows a graph (left side) and an enlarged region of the same graph (right side) to show detail for Bifidobacterium infantis growth in minimal media (no glucose), minimal media with glucose, and with GBX102 at various concentrations and time points. Percentage value concentrations shown are in reference to 15 mg/ml. Thus, 100% equals 15 mg/ml, 50% equals 7.5 mg/ml, etc.

FIG. 30 shows a schematic of the protocol for Example 3.

FIG. 31 provides the sample size and study arms for Example 3, Block 1.

FIG. 32 provides the sample size and study arms for Example 3, Block 2.

### DETAILED DESCRIPTION OF THE INVENTION

Compositions

Some aspects of the present invention are directed to a composition comprising a mixture of glycoproteins obtained from pig slime. “Pig slime” is mucus extracted from pig stomachs, usually in slaughterhouses, by extraction of the upper layer of the inner stomach tissue. In some embodiments, the composition is obtained without subjecting the mucins to conditions or reagents that release oligosaccharides from glycoproteins and glycopeptides. In some embodiments, the composition is obtained by a process comprising lowering the pH of the pig slime or a purified portion thereof to a pH between 2 and 5 to activate pepsin and cleave high molecular weight glycoproteins, and isolating the glycoproteins.

The term “glycoprotein” refers to proteins linked to oligosaccharides, e.g., proteins either N-linked or O-linked to oligosaccharides, and having a molecular weight of more than about 5 kDa. The term “glycopeptide” refers to peptides linked to oligosaccharides, e.g., peptides either N-linked or O-linked to oligosaccharides, and having a molecular weight of less than about 5 kDa. Methods of determining molecular weight of glycopeptides and glycoproteins are known in the art and are not limited. In some embodiments, the molecular weight of glycopeptides and glycoproteins are determined by size exclusion chromatography.

In some embodiments, peptides are defined as having a molecular weight of less than about 5 kDa. In some embodiments, the term peptides include glycopeptides. In some embodiments, proteins are defined as having a molecular weight of more than about 5 kDa. In some embodiments, the term proteins include glycoproteins.

In some embodiments, the oligosaccharide content of the composition is greater than about 10%, 15%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 50%, or 55% (w/w). As used herein, the oligosaccharide content is the total weight of oligosaccharide in the composition. Such weight does not include the weight of proteins or peptides attached to the oligosaccharides. In some embodiments, the oligosaccharide content of the composition is greater than about 30% (w/w). In some embodiments, the oligosaccharide content of the composition is greater than or equal to about 35% (w/w). In some embodiments, the oligosaccharide content of the composition is greater than or equal to about 40% (w/w). In some embodiments, the oligosaccharide content comprises substantially all oligosaccharides bound to glycoprotein or glycopeptide without substantially any unbound oligosaccharides. In some embodiments, the oligosaccharide content comprises substantially all oligosaccharides bound to glycoproteins without substantially any unbound oligosaccharides. Methods of determining oligosaccharide content are known in the art and are not limited. In some embodiments, oligosaccharide content is determined by HPAEC-PAD with an acid pre-treatment to hydrolyze the glycans into monosaccharides.

In some embodiments, the composition has a salt content of less than about 0.1%, 0.5%, 1%, 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3.0, 3.5%, or 4%. In some embodiments, the composition has a salt content of less than about 2%. In some embodiments, the salt content of the composition is substantially zero.

The composition may take the form of a slurry, powder, or liquid. In some embodiments, the composition is a powder and has a glycoprotein content of greater than about 65%, 67%, 70%, 71%, 72%, 73%, 74%, 75%, 76%, 77%, 78%, 79%, 80%, 82%, or 85% by weight. In some embodiments, the composition is a powder and has a glycoprotein content of greater than about 75% by weight. In some embodiments, the composition is a slurry and has a glycoprotein content of greater than about 65%, 67%, 70%, 71%, 72%, 73%, 74%, 75%, 76%, 77%, 78%, 79%, 80%, 82%, or 85% by weight. In some embodiments, the composition is a slurry and has a glycoprotein content of greater than about 60% by weight.

In some embodiments, the composition does not comprise more than about 5%, more than about 4%, more than about 3%, more than about 2%, more than about 1%, more than about 0.5%, or more than about 0.1% free glycans. In some embodiments, the composition has a free glycan content of less than 1%. In some embodiments, the composition has a free glycan content of substantially zero. The phrase “free glycans” refers to glycans that are not attached to a protein or polypeptide.

In some embodiments, the composition is obtained from pig slime, is a powder, and has an oligosaccharide content of greater than 30%, a free glycan content of less than 1%, and a glycoprotein content of greater than 75% by weight.

In some embodiments, the composition is for use as a medicament. In some embodiments, the composition is for use in a nutritional or dietary composition or nutritional or dietary premix. In some embodiments, the nutritional or dietary composition or nutritional or dietary premix is for use in supplementing an animal feed. In some embodiments, the nutritional or dietary composition or nutritional or dietary premix is for human consumption. In some embodiments, the nutritional or dietary composition or nutritional or dietary premix is for use as a food supplement. In some embodiments, the nutritional or dietary composition or nutritional or dietary premix is for use as a livestock (e.g. pig or poultry) or companion animal (e.g., dog or cat) feed supplement. The nutritional or dietary composition or nutritional or dietary premix may be in the form of a slurry, liquid, syrup, or powder.

In some embodiments, the composition is for use in a pharmaceutical or dietary composition further comprising a pharmaceutically acceptable carrier, diluent or excipient.

In some embodiments, the composition can be used for the preparation of nutritional/dietary supplement or complete food, in particular for oral delivery.

In some embodiments, the composition is in the form of nutritional supplement or complete food. In some embodiments, the composition is useful as an infant formula supplement. In some embodiments, the composition is useful as a human nutritional supplement. In some embodiments, the composition is useful as a domestic animal nutritional supplement. In some embodiments, the composition is useful as a dog or cat nutritional supplement. In some embodiments, the composition is useful as a livestock (e.g., pig, poultry) nutritional supplement.

The complete food or dietary/nutritional supplement according to the invention can be artificially enriched in vitamins, soluble or insoluble mineral salts or mixtures thereof or enzymes.

The compositions of the invention can be formulated as solid dosage forms containing a nutritional/dietary supplement with or without suitable excipients or diluents and prepared either by compression or molding methods well known in the art, encompassing compressed tablets and molded tablets or tablet triturates. In addition to the active or therapeutic/nutritional/cosmetic ingredient or ingredients, tablets contain a number or inert materials or additives, including those materials that help to impart satisfactory compression characteristics to the formulation, including diluents, binders, and lubricants. Other additives which help to give additional desirable physical characteristics to the finished tablet, such as disintegrators, coloring agents, flavoring agents, and sweetening agents might also be added in those compositions. In some embodiments, the solid dosage form is for use as a supplement for an animal (e.g., a dog or cat supplement, a pig supplement, a poultry supplement) or human.

As used herein, “diluents” are inert substances added to increase the bulk of the formulation to make the tablet a practical size for compression. Commonly used diluents include calcium phosphate, calcium sulfate, lactose, kaolin, mannitol, sodium chloride, dry starch, powdered sugar, silica, and the like. As used herein, “binders” are agents used to impart cohesive qualities to the powdered material. Binders, or “granulators” as they are sometimes known, impart cohesiveness to the tablet formulation, which insures the tablet remaining intact after compression, as well as improving the free-flowing qualities by the formulation of granules of desired hardness and size. Materials commonly used as binders include starch; gelatin; sugars, such as sucrose, glucose, dextrose, molasses, and lactose; natural and synthetic gums, such as acacia, sodium alginate, extract of Irish moss, panwar gum, ghatti gum, mucilage of isapol husks, carboxymethylcellulose, methylcellulose, polyvinylpyrrolidone, Veegum, microcrystalline cellulose, microcrystalline dextrose, amylose, and larch arabogalactan, and the like.

As used herein, “lubricants” are materials that perform a number of functions in tablet manufacture, such as improving the rate of flow of the tablet granulation, preventing adhesion of the tablet material to the surface of the dies and punches, reducing interparticle friction, and facilitating the ejection of the tablets from the die cavity. Commonly used lubricants include talc, magnesium stearate, calcium stearate, stearic acid, and hydrogenated vegetable oils.

As used herein, “disintegrators” or “disintegrants” are substances that facilitate the breakup or disintegration of tablets after administration. Materials serving as disintegrants have been chemically classified as starches, clays, celluloses, algins, or gums. Other disintegrators include Veegum HV, methylcellulose, agar, bentonite, cellulose and wood products, natural sponge, cation-exchange resins, alginic acid, guar gum, citrus pulp, cross-linked polyvinylpyrrolidone, carboxymethylcellulose, and the like.

As used herein, “coloring agents” are agents that give tablets a more pleasing appearance, and in addition help the manufacturer to control the product during its preparation and help the user to identify the product. Any of the approved certified water-soluble FD&C dyes, mixtures thereof, or their corresponding lakes may be used to color tablets. A color lake is the combination by adsorption of a water-soluble dye to a hydrous oxide of a heavy metal, resulting in an insoluble form of the dye.

As used herein, “flavoring agents” vary considerably in their chemical structure, ranging from simple esters, alcohols, and aldehydes to carbohydrates and complex volatile oils. Natural and synthetic flavors of almost any desired type are now available.

Further materials as well as formulation processing techniques and the like are set out in The Science and Practice of Pharmacy (Remington: The Science & Practice of Pharmacy), 22nd Edition, 2012, Lloyd, Ed. Allen, Pharmaceutical Press, which is incorporated herein by reference.

According to a particular aspect, the compositions according to the present invention are useful for use in infant food formulations or in premixes (which are then used to produce infant food formulations). The premix is usually in a dry form. The premix is usually produced by mixing the composition according to the present invention with other suitable ingredients, which are useful and/or essential in an infant formulation and/or premix (or which are useful and/or essential for the production of an infant formulation and/or premix).

According to a particular aspect, an infant formulation in the context of the present invention is usually a dry formulation, which is then dissolved either in water or in milk. The infant food premix or food formulations may further contain auxiliary agents, for example antioxidants (such as ascorbic acid or salts thereof, tocopherols (synthetic or natural); butylated hydroxytoluene (BHT); butylated hydroxyanisole (BHA); propyl gallate; tert-butyl hydroxyquinoline and/or ascorbic acid esters of a fatty acid); ethoxyquin, plasticizers, stabilizers (such as soy lecithin, citric acid esters of mono- and di-glycerides, and the like), humectants (such as glycerine, sorbitol, polyethylene glycol), dyes, fragrances, fillers and buffers.

According to a further aspect of the present invention, is provided an infant formula comprising a composition described herein for use in promoting, assisting or achieving balanced growth or development in an infant or preventing or reducing the risk of unbalanced growth or development in an infant

According to a particular aspect of the present invention, an infant formula may further comprise proteins fulfilling the minimum requirements for essential amino acid content and satisfactory growth, for example where over 50% by weight of the protein source is whey. Protein sources based on whey, casein and mixtures thereof may be used as well as protein sources based on soy. As far as whey proteins are concerned, the protein source may be based on acid whey or sweet whey (as readily available by-product of cheese making, preferably where caseino-glyco-macropeptide (CGMP) has been removed) or mixtures thereof and may include alpha-lactalbumin and beta-lactoglobulin in whatever desired proportions. According to a particular aspect of the present invention, an infant formula may further comprise a carbohydrate source such as lactose, saccharose, maltodextrin, starch and mixtures thereof. According to a particular aspect of the present invention, an infant formula may further comprise human milk oligosaccharides (HMOs).

According to a particular aspect of the present invention, an infant formula may further comprise a source of lipids including high oleic sunflower oil and high oleic safflower oil. The essential fatty acids linoleic and [alpha]-linolenic acid may also be added as may small amounts of oils containing high quantities of preformed arachidonic acid and docosahexaenoic acid such as fish oils or microbial oils. An infant formula may also contain all vitamins and minerals understood to be essential in the daily diet and in nutritionally significant amounts. Minimum requirements have been established for certain vitamins and minerals. Examples of minerals, vitamins and other nutrients optionally present in the infant formula include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chloride, potassium, sodium, selenium, chromium, molybdenum, taurine, and L-carnitine. An infant formula may optionally contain other substances which may have a beneficial effect such as fibres, lactoferrin, nucleotides, nucleosides, and the like.

In some embodiments, the composition is in the form of a liquid (e.g., water, milk, a sugar solution, etc.) suitable for oral administration to an infant, including a newborn.

In some embodiments, the composition is for use in prevention and/or treatment of an unbalance of the microbiota and/or disorders associated with dysbiosis such as asymptomatic dysbiotic microbiota. The term “dysbiosis” is defined as a state in which the microbiota produces harmful effects via (a) qualitative and quantitative changes in the content or amount of the microbiota itself, (b) changes in their metabolic activities; and/or (c) changes in their local distribution. Abnormalities in microbiota composition and activity (called dysbiosis) have been implicated in the emergence of the metabolic syndrome, which include diseases such as obesity, type 2 diabetes and cardiovascular diseases. In some embodiments, a human with dysbiosis exhibits insulin resistance or obesity. In some embodiments, the composition is for use in prevention and/or treatment of obesity. In some embodiments, the composition is for use in weight control.

In some embodiments, the composition is capable of inhibiting glycan-mediated binding of one or more pathogenic micro-organisms to mucosal cells when orally administered to a subject. As used herein, a subject is a mammal. In preferred embodiments, the subject is a human, cat, or dog. In other embodiments, the animal is a livestock animal. Many pathogens like bacteria, viruses and protozoan parasites, express lectins to attach to the glycans of the epithelial cell surface of the host and colonize or invade the host and cause disease. The compositions disclosed herein may have structures similar to surface glycans of intestinal epithelial cells (i.e., epithelial cells of the gut). Thus, the glycoproteins of the present compositions may serve as bacterial lectin ligand analogs blocking bacterial attachment and act as antiadhesive antimicrobials. Thus, the glycoproteins of the present compositions may therefore serve as soluble decoy moieties to prevent pathogen binding and decrease the risk of infections as unbound pathogens are carried downstream and excreted with the feces. Alternatively, Thus, the glycoproteins of the present compositions may bind receptors such as lectins on host cells which could block pathogen binding to host cells via a competition mechanism, reducing risk of infections. The aforementioned mechanisms are not only relevant to gastrointestinal tract environment but also to other body locations that contain mucus such as, but not limited to, the respiratory or urinary tract.

In some embodiments, the one or more pathogenic microorganisms comprise Escherichia coli, Helicobacter pylori, Streptococcus spp., Toxoplasma gondii, Plasmodium falciparum, Clostridium spp., Salmonella spp., influenza virus, rotavirus, and respirovirus. In some embodiments, administration of the composition inhibits glycan-mediated binding of one or more pathogenic micro-organisms to mucosal cells by about 5%, 10%, 20%, 30%, 40%, 50%, 60%, 70%, 80%, 85%, 90%, 95%, 99%, 99.9% or more. In some embodiments, administration of the composition inhibits glycan-mediated binding of one or more pathogenic micro-organisms to mucosal cells by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more.

In some embodiments, the composition is capable of reducing the growth of one or more pathogenic microorganisms in the gut when administered to a subject. In some embodiments, the one or more pathogenic microorganisms comprise Escherichia coli, Helicobacter pylori, Streptococcus spp., Toxoplasma gondii, Plasmodium falciparum, Clostridium spp., Salmonella spp., influenza virus, rotavirus, and respirovirus. In some embodiments, administration of the composition inhibits the growth of the pathogenic microorganism by about 5%, 10%, 20%, 30%, 40%, 50%, 60%, 70%, 80%, 85%, 90%, 95%, 99%, 99.9% or more. In some embodiments, administration of the composition inhibits the growth of the pathogenic microorganism by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more.

In some embodiments, the composition is capable of reducing the level of one or more pathogenic microorganisms in the gut when administered to a subject. In some embodiments, the one or more pathogenic microorganisms comprise Escherichia coli, Helicobacter pylori, Streptococcus spp., Toxoplasma gondii, Plasmodium falciparum, Clostridium spp., Salmonella spp., influenza virus, rotavirus, and respirovirus. In some embodiments, administration of the composition reduces the level of the pathogenic microorganism in the gut by about 5%, 10%, 20%, 30%, 40%, 50%, 60%, 70%, 80%, 85%, 90%, 95%, 99%, 99.9% or more. In some embodiments, administration of the composition reduces the level of the pathogenic microorganism in the gut by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more. In some embodiments, the composition is capable of reducing the level of Escherichia coli (e.g., pathogenic Escherichia coli) in the gut when administered to a subject. In some embodiments, administration of the composition reduces the level of Escherichia coli in the gut by about 10%-80%, 20%-70%, 30%-60%, or any range therebetween.

In some embodiments, the composition is capable of increasing the growth of one or more commensal microorganisms in the gut when administered to a subject. In some embodiments, the one or more (e.g., 1, 2, 3, 4, 5, or all 6 of) commensal microorganisms comprise Lactobacillus acidophilus, Lactobacillus reuteri, Akkermansia mucimphila, Bacteroides thetaiotaomicron, Bifidobacterium breve, and/or Bifidobacterium infantis. In some embodiments, administration of the composition enhances or increases the growth of the one or more commensal microorganisms by about 5%, 10%, 20%, 30%, 40%, 50%, 60%, 70%, 80%, 85%, 90%, 95%, 99%, 99.9% or more. In some embodiments, administration of the composition enhances or increases the growth of the one or more commensal microorganisms by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more. In some embodiments, the one or more commensal microorganisms is Lactobacillus acidophilus. In some embodiments, the one or more commensal microorganisms is Lactobacillus acidophilus. In some embodiments, the one or more commensal microorganisms is Lactobacillus reuteri. In some embodiments, the one or more commensal microorganisms is Akkermansia mucimphila. In some embodiments, the one or more commensal microorganisms is Bacteroides thetaiotaomicron. In some embodiments, the one or more commensal microorganisms is Bifidobacterium breve. In some embodiments, the one or more commensal microorganisms is Bifidobacterium infant's. In some embodiments, increasing or enhancing commensal microorganism level or activity reduces inflammation, increases lactate production in the gut, increases short-chain fatty acid (SCFA) production in the gut, lowers pH in the gut, and/or inhibits growth of one or more pathogenic microorganisms (e.g., Escherichia coli, Helicobacter pylori, Streptococcus spp., Toxoplasma gondii, Plasmodium falciparum, Clostridium spp., Salmonella spp., influenza virus, rotavirus, and/or respirovirus) in the gut.

In some embodiments, the composition is capable of reducing inflammation when orally administered to a subject. In some embodiments, administration of the composition reduces inflammation (e.g., inflammation in the gut) by about 5%, 10%, 20%, 30%, 40%, 50%, 60%, 70%, 80%, 85%, 90%, 95%, 99%, 99.9% or more. In some embodiments, administration of the composition reduces inflammation (e.g., inflammation in the gut) by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more. In some embodiments, reducing inflammation comprises a reduction in calprotectin in the blood stream or stool of the subject. In some embodiments, calprotectin is increased in the stool or decreased in the blood by about 5%, 10%, 20%, 30%, 40%, 50%, 60%, 70%, 80%, 85%, 90%, 95%, 99%, 99.9% or more. In some embodiments, calprotectin is increased in the stool or decreased in the blood by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more.

In some embodiments, the composition is capable of increasing lactate production in the gut when orally administered to a subject. In some embodiments, lactate production is increased by about 5%, 10%, 20%, 30%, 40%, 50%, 60%, 70%, 80%, 85%, 90%, 95%, 99%, 99.9% or more. In some embodiments, lactate production is increased by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more.

In some embodiments, the composition, when orally administered to a subject, is capable of increasing short-chain fatty acid (SCFA) production in the gut of the subject. SCFAs play an important role in host health and SCFA production is considered a benefit to the host. SCFAs serve as a source of energy for intestinal epithelial cells, and help maintain intestinal integrity by promoting mucus production and gut barrier function. SCFAs also have anti-tumor effects on colonic carcinoma. SCFAs have further been shown to have immunomodulation effects including T cell regulation and intestinal anti-inflammatory properties. Moreover, SCFAs are involved in the modulation of homeostasis and metabolism including reduction of cholesterol and fatty acid synthesis in the liver. SCFAs have also been shown to have antibacterial properties via stimulating antimicrobial peptides and reducing luminal pH. In some embodiments, SCFAs comprise at least one of butyrate and propionate.

In some embodiments, SCFA production (e.g., butyrate and/or propionate) is increased by about 5%, 10%, 20%, 30%, 40%, 50%, 60%, 70%, 80%, 85%, 90%, 95%, 99%, 99.9% or more. In some embodiments, SCFA production (e.g., butyrate and/or propionate) is increased by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more.

In some embodiments, the composition, when orally administered to a subject, is capable of lowering pH in the gut of the subject. Lowering the pH is advantageous as growth and viability of beneficial bacteria can be enhanced. In some embodiments, the decrease in pH is caused by an increase in SCFA production in the gut.

Methods of Manufacture

Some aspects of the present disclosure are directed to a method of manufacturing a composition comprising a mixture of glycopeptides, comprising the following steps: providing a composition comprising mucins from the outer mucus layer of pig stomach (i.e., pig slime) or a partially purified fraction thereof and water; adjusting the pH of the composition to 2 to 5 (e.g., 3.0 to 3.5) with the addition of an acid and incubating the solution to hydrolyze the composition; and isolating a composition comprising glycopeptides from the hydrolyzed composition. Some aspects of the present disclosure are directed to a method of manufacturing a composition comprising a mixture of glycopeptides, comprising the following steps: (a) providing a composition comprising mucins from the outer mucus layer of pig stomach (i.e., pig slime) or a partially purified fraction thereof and water; (b) adjusting the pH of the composition to 2 to 5 (e.g., 3.0 to 3.5) with the addition of an acid and incubating the solution to hydrolyze the composition; (c) isolating an aqueous phase from the composition; (d) defatting the isolated aqueous phase; and (e) precipitating and isolating a composition comprising glycopeptides from the defatted aqueous phase.

In some embodiments, the composition comprising mucins from the outer mucus layer of pig stomach (i.e., pig slime) or a partially purified fraction thereof comprises one or more proteases (e.g., pepsin) having proteolytic activity at a pH of between 2-5. In some embodiments, the composition comprising mucins from the outer mucus layer of pig stomach (i.e., pig slime) or a partially purified fraction thereof has not been subject to conditions or reagents that release oligosaccharides from glycoproteins and glycopeptides.

In some embodiments, the method further comprises a step (f) of dewatering the isolated composition. In some embodiments, the method further comprises a step (g) of drying the dewatered composition to obtain a composition comprising a mixture of glycopeptides.

In some embodiments, the composition comprising a mixture of glycopeptides (i.e., the composition obtained by the methods described herein) has an oligosaccharide content of greater than or equal to about 44% (w/w), greater than or equal to about 40% (w/w), greater than or equal to about 38% (w/w), greater than or equal to about 36% (w/w), greater than or equal to about 34% (w/w), greater than or equal to about 32% (w/w), greater than or equal to about 31% (w/w), greater than or equal to about 30% (w/w), greater than or equal to about 29.5% (w/w), greater than or equal to about 29% (w/w), greater than or equal to about 28.5% (w/w), greater than or equal to about 28% (w/w), greater than or equal to about 27% (w/w), greater than or equal to about 26% (w/w), greater than or equal to about 25% (w/w), greater than or equal to about 24% (w/w), or greater than or equal to about 20% (w/w). In some embodiments, the composition comprising a mixture of glycopeptides has an oligosaccharide content of greater than or equal to about 30% (w/w).

In some embodiments, the composition of step a) has been homogenized. Methods of homogenization are not limited and may be any suitable method known in the art. In some embodiments, the composition is homogenized with a blender (e.g., for 1-2 minutes). In some embodiments, the composition is homogenized by sonication.

The ratio of pig stomach outer mucus layer (e.g., pig slime) to water for the composition of step (a) is not limited and may be any suitable ratio to enable processing of the mucus by the methods disclosed herein. In some embodiments, no water is added to the pig stomach outer mucus layer. In some embodiments, the composition of step a) comprises about a 1:1 ratio of pig stomach outer mucus layer to water.

The method of adjusting the pH of the composition in step b) is not limited. In some embodiments, the pH is adjusted in step b) with HCl. Any suitable time and temperature combination may be used to cleave high molecular weight glycopeptides in step b) via proteases in the composition (e.g., pepsin), as long as such conditions do not release or substantially release oligosaccharides from glycoproteins and glycopeptides of the mucins in the pig stomach outer mucus layer. In some embodiments, the pH is adjusted to a pH of between 2 and 5. In some embodiments, the composition is incubated in step b) at a pH of 2.8 to 3.7 for 1-5 hours at 40-50° C. In some embodiments, the composition is incubated in step b) at a pH of 3.0 to 3.5 for 2-4 hours at 45° C. In some embodiments, the incubation comprises shaking.

In some embodiments, step b) further comprises a further final addition of acid after incubation. In some embodiments, step b) further comprises adding 1 part of an aqueous solution having a pH of 3.0 to 3.5 to 2-3 parts of the composition after incubation.

The isolation of the aqueous phase in step c) may be by any suitable method known in the art and is not limited. In some embodiments, the aqueous phase is isolated in step c) by a process comprising centrifugation followed by removal of the aqueous phase. Any suitable centrifugation speed may be used that separates the aqueous phase. In some embodiments, centrifugation is at 500 to 10,000×g. In some embodiments, centrifugation is performed at 4° C. In some embodiments, centrifugation is performed at 3000-4000 rpm (e.g., 3500 rpm).

In some embodiments, the aqueous phase obtained in step c) is decanted and further processed to remove insoluble material. In some embodiments, the insoluble material is removed by centrifugation. In some embodiments, the aqueous phase obtained in step c) is filtered to remove insoluble material prior to step d). In some embodiments, the filtration is performed with a cloth filter. In some embodiments, the cloth filter has a pore size of about 100-200 μM.

The method of defatting in step d) is not limited and may be any suitable method in the art. In some embodiments, defatting is with a non-polar solvent. In some embodiments, the isolated aqueous phase is defatted in step d) by the addition of about 5% v/w heptane or hexane followed by incubation for 6-18 hours at 30° C. and removal of the heptane or hexane phase. In some embodiments, the defatted aqueous phase is processed to remove insoluble materials prior to step d). In some embodiments, the defatted aqueous phase is filtered to remove insoluble material prior to step e). In some embodiments, the filtration is performed with a cloth filter. In some embodiments, the cloth filter has a pore size of about 100-200 μM.

In some embodiments, the defatted aqueous phase is concentrated to ½ to ¼ of the initial volume prior to step e). In some embodiments, the defatted aqueous phase is concentrated to ⅓ of the initial volume prior to step e). Any suitable method of concentration may be used and is not limited. In some embodiments, concentration is with a rotary evaporator. In some embodiments, the defatted aqueous phase is concentrated to ½ to ¼ of the initial volume using a rotary evaporator at 60° C. In some embodiments, the concentrated composition is incubated for 8-24 hours at about 4° C. to allow settling.

In some embodiments, the defatted aqueous phase (e.g., defatted and concentrated aqueous phase) is precipitated with an organic solvent in step e). Any suitable organic solvent may be used and is not limited. In some embodiments, the organic solvent is ethanol or acetone. In some embodiments, the organic solvent (e.g., acetone, ethanol) is added at a ratio of about 60%, 65%, 70%, 75%, 80%, 85%, 90%, or 95% (v/v) to the defatted aqueous phase. In some embodiments, the organic solvent (e.g., acetone, ethanol) is added at a ratio of 80% (v/v) to the defatted aqueous phase. In some embodiments, the organic solvent (e.g., acetone, ethanol) is added at a ratio of 80% (v/v) to the defatted aqueous phase. In some embodiments the defatted aqueous phase (e.g., defatted and concentrated aqueous phase) is precipitated with an organic solvent (e.g., acetone, ethanol) at about 4° C. In some embodiments, the precipitant is isolated in step e) by filtration or centrifugation. Any suitable methods of filtration or centrifugation may be used and are not limited. In some embodiments, the precipitant is isolated by centrifugation at 500 to 10,000 g. In some embodiments, centrifugation is performed at 3000-4000 rpm (e.g., 3500 rpm).

In some embodiments, the supernatant obtained after isolation of the precipitant is further treated with an aqueous solvent to further precipitate glycopeptides and glycoproteins. In some embodiments, the further precipitation is carried out with a ratio of about 90% (v/v) ethanol or acetone at 4 4° C. In some embodiments, the precipitant is isolated by centrifugation or filtration and pooled with the previously obtained precipitant.

The method of dewatering in step f) is not limited and may be any suitable method known in the art. In some embodiments, the isolated composition (precipitant) is dewatered with an organic solvent. In some embodiments, the composition is dewatered in step 0 with ethanol.

The method of drying the dewatered composition of step g) is not limited and may be any suitable method known in the art. In some embodiments, drying the dewatered composition of step g) comprises freeze drying or rotary evaporation. In some embodiments, the dried composition is milled or homogenized to provide a powder.

Some aspects of the present invention are directed to a composition obtained by the methods disclosed herein. In some embodiments, the composition obtained by the methods disclosed herein are compositions as described herein. In some embodiments, the composition is a powder and has an oligosaccharide content of greater than 30%, a free glycan content of less than 1%, and a glycoprotein content of greater than 75% by weight.

Methods of Use

The present compositions are much more structurally diverse than previous pre-biotic formulations containing fructooligosaccharides (FOS) and/or galactoligosaccharides (GOS). FOS and GOS are linear chain, simpler oligosaccharides that do not contain the structural complexity and diversity of the present composition. Unlike these previous prebiotics, the glycoprotein- or glycopeptide-bound oligosaccharides, or only glycopeptide-bound oligosaccharides, of the present composition have multiple building blocks, branched structures and a higher variety of structures which impart biological functionalities including anti-microbial activity, better microbiota maintenance, and immunological activity.

Some aspects of the present invention are related to a method of treating, preventing, or reducing the severity of a pathogenic microorganism infection of the gut of a subject comprising orally administering to the subject a composition disclosed herein or a composition manufactured by a method disclosed herein. In some embodiments, the pathogenic microorganism is selected from Escherichia coli, Helicobacter pylori, Streptococcus spp., Toxoplasma gondii, Plasmodium falciparum, Clostridium spp., Salmonella spp., influenza virus, rotavirus, and respirovirus. In some embodiments, administration of the composition inhibits glycan-mediated binding of one or more pathogenic micro-organisms to mucosal cells by about 5%, 10%, 20%, 30%, 40%, 50%, 60%, 70%, 80%, 85%, 90%, 95%, 99%, 99.9% or more. In some embodiments, administration of the composition inhibits glycan-mediated binding of one or more pathogenic micro-organisms to mucosal cells by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more. In some embodiments, administration of the composition to a patient inhibits growth or decreases the level of one or more pathogenic microorganisms (e.g., Escherichia coli) in the gut of the patient by about 5%, 10%, 20%, 30%, 40%, 50%, 60%, 70%, 80%, 85%, 90%, 95%, 99%, 99.9% or more. In some embodiments, administration of the composition to a patient inhibits growth or decreases the level of one or more pathogenic microorganisms (e.g., pathogenic Escherichia coli) in the gut of the patient by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold or more.

Some aspects of the present invention are related to a method of reducing the fat mass of a subject comprising orally administering to the subject a composition disclosed herein or a composition manufactured by a method disclosed herein.

Some aspects of the present invention are related to a method of treating, preventing, or reducing inflammation in a subject comprising orally administering to the subject a composition disclosed herein or a composition manufactured by a method disclosed herein. In some embodiments, administration of the composition reduces inflammation (e.g., inflammation in the gut) by about 5%, 10%, 20%, 30%, 40%, 50%, 60%, 70%, 80%, 85%, 90%, 95%, 99%, 99.9% or more. In some embodiments, administration of the composition reduces inflammation (e.g., inflammation in the gut) by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more. In some embodiments, reduces a level of calprotectin in the blood stream or stool of the subject. In some embodiments, calprotectin is decreased in the stool or decreased in the blood by about 5%, 10%, 20%, 30%, 40%, 50%, 60%, 70%, 80%, 85%, 90%, 95%, 99%, 99.9% or more. In some embodiments, calprotectin is decreased in the stool or blood by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more (e.g., compared to before administration of a composition of the invention).

Some aspects of the present invention are related to a method of increasing production of short chain fatty acid (SCFA) (e.g., butyrate and/or propionate) in the gut of a subject comprising orally administering to the subject a composition disclosed herein or a composition manufactured by a method disclosed herein. In some embodiments, SCFA production is increased by about 5%, 10%, 20%, 30%, 40%, 50%, 60%, 70%, 80%, 85%, 90%, 95%, 99%, 99.9% or more. In some embodiments, SCFA production is increased by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more. In some embodiments, the composition, when orally administered to a subject, is capable of lowering pH in the gut of the subject. In some embodiments, the decrease in pH is caused by an increase in SCFA production in the gut.

In some embodiments, administration of the composition to a patient increases growth or increases the level of one or more commensal bacteria (e.g., Lactobacillus acidophilus, Lactobacillus reuteri, Akkermansia muciniphila, Bacteroides thetaiotaomicron, Bifidobacterium breve, and/or Bifidobacterium infantis) in the gut of the patient by about 5%, 10%, 20%, 30%, 40%, 50%, 60%, 70%, 80%, 85%, 90%, 95%, 99%, 99.9% or more. In some embodiments, administration of the composition to a patient increases growth or increases the level of one or more commensal bacteria (e.g., Lactobacillus acidophilus, Lactobacillus reuteri, Akkermansia muciniphila, Bacteroides thetaiotaomicron, Bifidobacterium breve, and/or Bifidobacterium infantis) in the gut of the patient by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold or more.

Some aspects of the present invention are related to a method of improving gut barrier integrity in the gut of a subject comprising orally administering to the subject a composition disclosed herein or a composition manufactured by a method disclosed herein.

Some aspects of the present invention are related to a method of assisting the development of beneficial gut microbiota in an infant comprising orally administering to the infant a composition disclosed herein or a composition manufactured by a method disclosed herein. In some embodiments, the infant is a newborn. In some embodiments, the newborn was delivered by caesarean section (C-section). In some embodiments, the beneficial gut microbiota comprises one or more of Lactobacillus acidophilus, Lactobacillus reuteri, Akkermansia muciniphila, Bacteroides thetaiotaomicron, Bifidobacterium breve, and/or Bifidobacterium infantis. In some embodiments, the beneficial gut microbiota includes a decreased level of a pathogenic microorganism. In some embodiments, the pathogenic microorganism is one or more of Escherichia coli, Helicobacter pylori, Streptococcus spp., Toxoplasma gondii, Plasmodium falciparum, influenza virus, rotavirus, and/or respirovirus.

The subject (also sometimes referred to as a patient throughout) is not limited. In some embodiments, the subject is a human. In some embodiments, the subject may be an infant (1 years old or less for a human), a toddler (3 years old or less for a human), a child, a young adult, an adult or a geriatric. In some embodiments, the infant is a newborn. In some embodiments, the newborn was delivered by caesarean section (C-section). The subject may be male or female. In some embodiments, the subject is female and of child-bearing age. In some embodiments, the subject has cancer. In some embodiments, the subject has systemic inflammation. In some embodiments, the subject has acute colitis. In some embodiments, the subject has inflammatory bowel disease.

Some aspects of the present disclosure are related to a method of treating a cancer in a subject in need thereof, comprising administering to the subject a composition described herein or a composition made by a method described herein.

The subject is not limited and may be any subject described herein. In some embodiments, the subject is human.

The cancer is not limited and may be any suitable cancer. In some embodiments, the cancer is a solid tumor cancer. In some embodiments, the cancer is adrenal cancer, biliary cancer, bladder cancer, brain cancer, breast cancer, cervical cancer, colon cancer, colorectal cancer, rectum cancer, endometrial cancer, esophageal cancer, head or neck cancer, kidney cancer, liver cancer, non-small cell lung cancer, lung cancer, lymphoma, melanoma, meninges cancer, non-melanoma skin cancer, ovarian cancer, pancreatic cancer, prostate cancer, sarcoma, small intestine cancer, or stomach cancer. In some embodiments, the cancer is melanoma or colorectal cancer.

In some embodiments, the cancer is an immunotherapy responsive cancer. In some embodiments, the cancer is a checkpoint inhibitor responsive cancer. The checkpoint inhibitor is not limited and may be any suitable checkpoint inhibitor. In some embodiments, the checkpoint inhibitor is an antibody against at least one checkpoint protein, e.g., PD-1, CTLA-4, PD-L1 or PD-L2. In some embodiments, the checkpoint inhibitor is an antibody that is effective against two or more of the checkpoint proteins selected from the group of PD-1, CTLA-4, PD-L1 and PD-L2. In some embodiments, the checkpoint inhibitor is a small molecule, non-protein compound that inhibits at least one checkpoint protein. In one embodiment, the checkpoint inhibitor is a small molecule, non-protein compound that inhibits a checkpoint protein selected from the group consisting of PD-1, CTLA-4, PD-L1 and PD-L2. In some embodiments, the checkpoint inhibitor administered is nivolumab (Opdivo®, BMS-936558, MDX1106, commercially available from BristolMyers Squibb, Princeton N.J.), pembrolizumab (Keytruda® MK-3475, lambrolizumab, commercially available from Merck and Company, Kenilworth N.J.), atezolizumab (Tecentriq®, Genentech/Roche, South San Francisco Calif.), durvalumab (MEDI4736, Medimmune/AstraZeneca), pidilizumab (CT-011, CureTech), PDR001 (Novartis), BMS-936559 (MDX1105, BristolMyers Squibb), avelumab (MSB0010718C, Merck Serono/Pfizer), or SHR-1210 (Incyte). Additional antibody PD1 pathway inhibitors for use in the methods described herein include those described in U.S. Pat. No. 8,217,149 (Genentech, Inc) issued Jul. 10, 2012; U.S. Pat. No. 8,168,757 (Merck Sharp and Dohme Corp.) issued May 1, 2012, U.S. Pat. No. 8,008,449 (Medarex) issued Aug. 30, 2011, and U.S. Pat. No. 7,943,743 (Medarex, Inc) issued May 17, 2011. In some embodiments, the checkpoint inhibitor is an anti-PD-1 antibody, an anti-CTLA-4 antibody, an anti-PD-L1 antibody, or an anti-PD-L2. In some embodiments, the checkpoint inhibitor is nivolumab, pembrolizumab, atezolizumab, durvalumab, pidilizumab, PDR001, BMS-936559, avelumab, or SHR-1210. In some embodiments, the checkpoint inhibitor is nivolumab.

In some embodiments, the subject is further administered the checkpoint inhibitor. In some embodiments, the subject is periodically administered the composition starting at least 1 day, at least 2 days, at least 4 days, at least 1 week, at least 10 days, at least 2 weeks, at least 3 weeks, at least 1 month, at least 6 weeks, at least 2 months, at least 3 months, or at least 4 months prior to administration of the checkpoint inhibitor. In some embodiments, the subject is periodically administered the composition at the same time as the checkpoint inhibitor or starting about at least 1 day, at least 2 days, at least 4 days, at least 1 week, at least 10 days, at least 2 weeks, at least 3 weeks, at least 1 month, at least 6 weeks, at least 2 months, at least 3 months, or at least 4 months after administration of the checkpoint inhibitor. In some embodiments, periodic administration comprises administration at least once per day, at least once every other day, or at least once every three days.

Methods of administering the composition are not limited and may be any suitable method, or method disclosed herein. In some embodiments, the composition is orally administered.

In some embodiments, administration of the composition increases microbiota diversity in the gut of the subject. In some embodiments, administration of the composition increases the growth of one or more commensal bacteria. The commensal bacteria are not limited and may be any described herein and by any amount described herein. In some embodiments, administration of the composition increases production of SCFA, decreases pH, improves gut barrier integrity, and/or assists the development of beneficial gut microbiota as described throughout the specification.

In some embodiments, administration of the composition increases infiltration of immune cells into tumors. In some embodiments, infiltration is increased by 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more In some embodiments, the immune cells are selected from CD4+ T-cells; IFN-γ+, Foxp3+ T-cells; CD8+ T-cells; dendritic cells; plasmacytoid dendritic cells; B cells; macrophages; and natural-killer cells.

In some embodiments, administration of the composition reduces systemic inflammation in the subject. In some embodiments, systemic inflammation is decreased by about 5%, 10%, 20%, 30%, 40%, 50%, 60%, 70%, 80%, 85%, 90%, 95%, 99%, 99.9% or more. In some embodiments, a systemic level of one or more inflammatory cytokines is reduced. In some embodiments, the inflammatory cytokines are selected from Eotaxin, G-CSF, GM-CSF, IFN-γ, IL-1α, IL-1β, IL-2, IL-3, IL-4, IL-5, IL-6, IL-9, IL-10, IL-12, IL-13, IL-17A, KC, MCP-1 (MCAF), MIP-1α, MIP-1β, RANTES, or TNF-α. In some embodiments, the level of the inflammatory cytokine is decreased by about 5%, 10%, 20%, 30%, 40%, 50%, 60%, 70%, 80%, 85%, 90%, 95%, 99%, 99.9% or more. In some embodiments, administration of the composition increases cancer cell death. In some embodiments, cancer cell death is increased by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more. In some embodiments, increased cancer cell death is detected by increased inosine in the subject.

In some embodiments, administration of the composition increases production of short chain fatty acid (SCFA) in the gut of the subject. In some embodiments, SCFA is increased by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more. In some embodiments, administration of the composition reduces the side effects of an anti-cancer therapy administered to the subject.

Some aspects of the present disclosure are related to a method of treating inflammation in a subject in need thereof, comprising administering to the subject a composition described herein or a composition made by a method described herein. In some embodiments, administration of the composition reduces inflammation (e.g., inflammation in the gut, systemic inflammation, inflammation caused by a disease or condition, inflammation associated with inflammatory bowel disease, inflammation associated with acute colitis) by about 5%, 10%, 20%, 30%, 40%, 50%, 60%, 70%, 80%, 85%, 90%, 95%, 99%, 99.9% or more. In some embodiments, administration of the composition reduces inflammation (e.g., inflammation in the gut, systemic inflammation, inflammation caused by a disease or condition, inflammation associated with inflammatory bowel disease, inflammation associated with acute colitis) by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more. In some embodiments, reducing inflammation comprises a reduction in calprotectin in the blood stream or stool of the subject. In some embodiments, calprotectin is increased in the stool or decreased in the blood by about 5%, 10%, 20%, 30%, 40%, 50%, 60%, 70%, 80%, 85%, 90%, 95%, 99%, 99.9% or more. In some embodiments, calprotectin is increased in the stool or decreased in the blood by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more.

In some embodiments, the subject has an inflammatory disease or condition. In some embodiments, the inflammatory disease or condition is inflammatory bowel disease or acute colitis.

In some embodiments, administration of the composition increases microbiota diversity in the gut of the subject. In some embodiments, administration of the composition increases the growth of one or more commensal bacteria. The commensal bacteria are not limited and may be any described herein and by any amount described herein. In some embodiments, administration of the composition increases production of SCFA, decreases pH, improves gut barrier integrity, and/or assists the development of beneficial gut microbiota as described throughout the specification.

In some embodiments, the composition is administered at least once per day, at least once every other day, at least once every three days, at least once every four days, at least once every five days, at least once every week.

Methods of administering the composition are not limited and may be any suitable method, or method disclosed herein. In some embodiments, the composition is orally administered.

In some embodiments, a systemic level of one or more inflammatory cytokines is reduced. In some embodiments, the inflammatory cytokines are selected from Eotaxin, G-CSF, GM-CSF, IFN-γ, IL-1α, IL-1β, IL-2, IL-3, IL-4, IL-5, IL-6, IL-9, IL-10, IL-12, IL-13, IL-17A, KC, MCP-1 (MCAF), MIP-1α, MIP-1β, RANTES, or TNF-α. In some embodiments, the level of the inflammatory cytokine is decreased by about 5%, 10%, 20%, 30%, 40%, 50%, 60%, 70%, 80%, 85%, 90%, 95%, 99%, 99.9% or more.

In some embodiments, administration of the composition increases production of short chain fatty acid (SCFA) in the gut of the subject. In some embodiments, SCFA is increased by about 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 10-fold, 20-fold, 50-fold, 100-fold, or more.

The subject is not limited and may be any subject disclosed herein. In some embodiments, the subject is human.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or prior publication, or for any other reason. All statements as to the date or representation as to the contents of these documents is based on the information available to the applicants and does not constitute any admission as to the correctness of the dates or contents of these documents.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more active agents, additives, ingredients, optional agents, types of organism, disorders, subjects, or combinations thereof, can be excluded.

Where the claims or description relate to a composition of matter, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

“Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

Specific examples of certain aspects of the inventions disclosed herein are set forth below in the Examples.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

### EXAMPLES

### Example 1—Production of GBX102

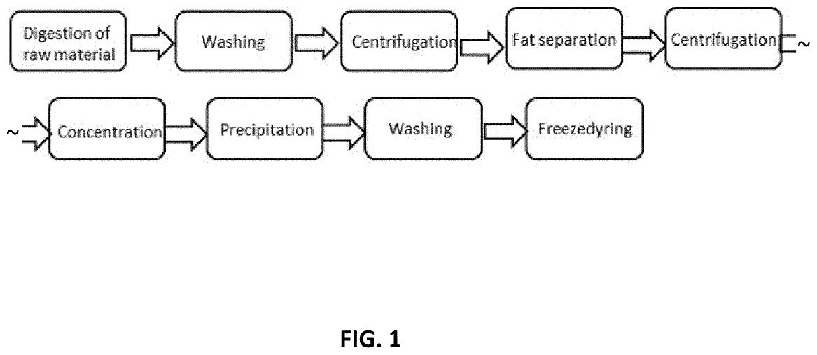

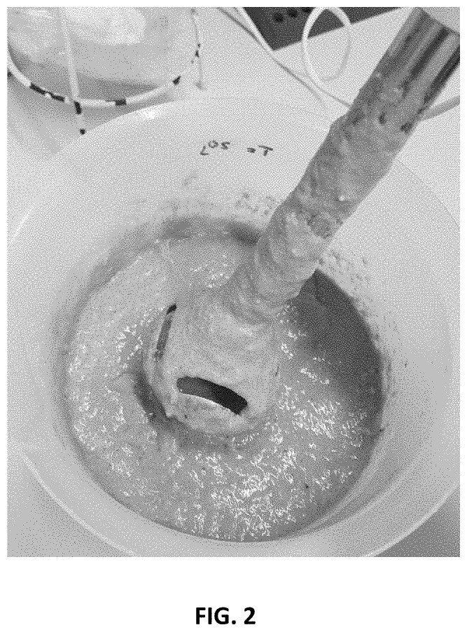

A sample of slime from pig stomachs was obtained. This sample contained slime, fat and meaty substances. The sample was split into two replicates and each processed according to the process set forth in FIG. 1. 600 g of sample was homogenized with a hand blender for 1-2 min. FIG. 2 shows the sample after homogenization.

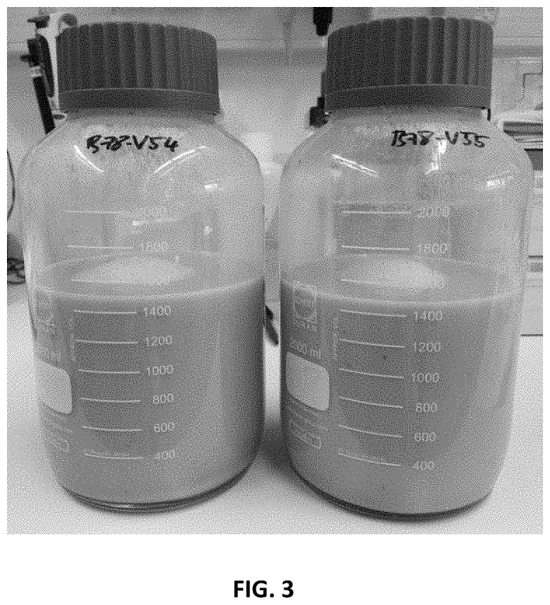

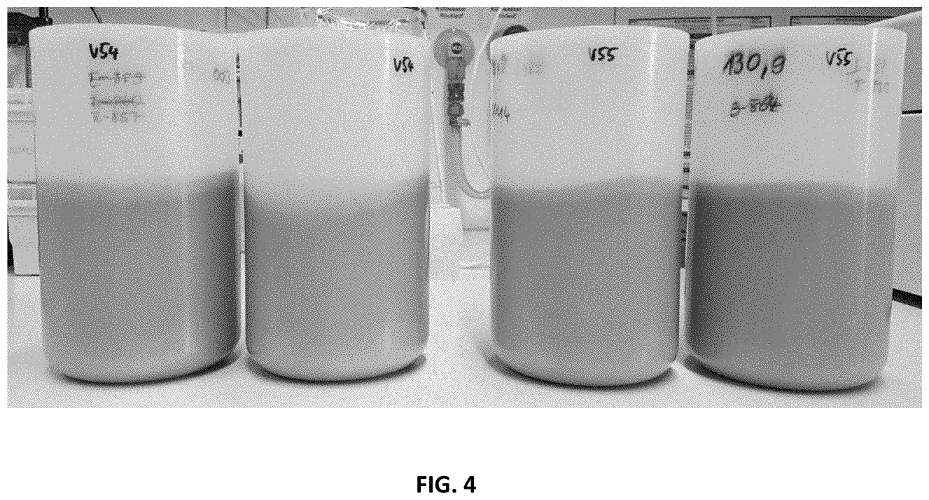

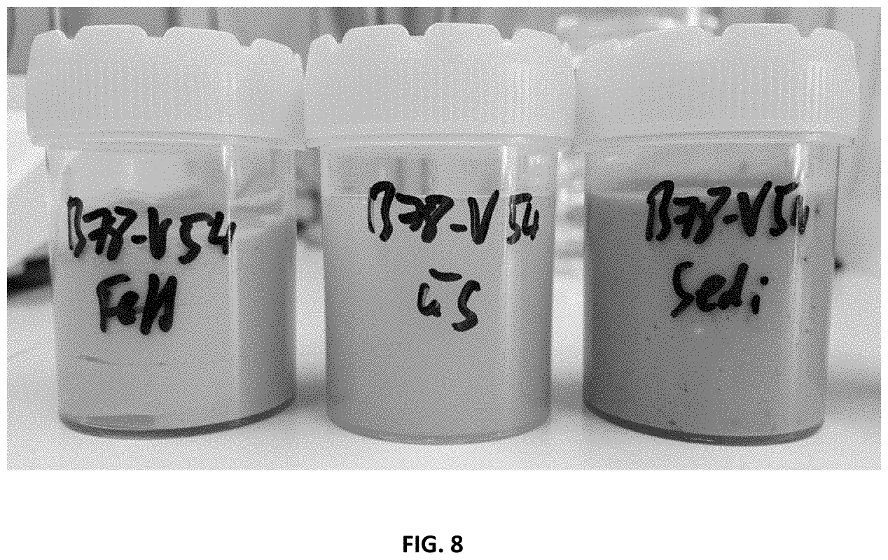

The resulting homogenate was hydrolyzed by addition of 600 g of H₂O and pH adjustment to 3.0-3.5 with 2M HCl. The resulting suspension was incubated for 3 h at 45° C. on a shaker. At the end of the incubation time 400 g of HCl solution at pH 3.0-3.5 was added. The resulting solution for each replicate is shown in FIG. 3. The solution from acid hydrolysis was centrifuged at 3500 rpm and 4° C., and the different phases (fat, aqueous and sediment) were isolated. The resulting solutions separated into different phases for each replicate are shown in FIG. 4 and FIG. 8.

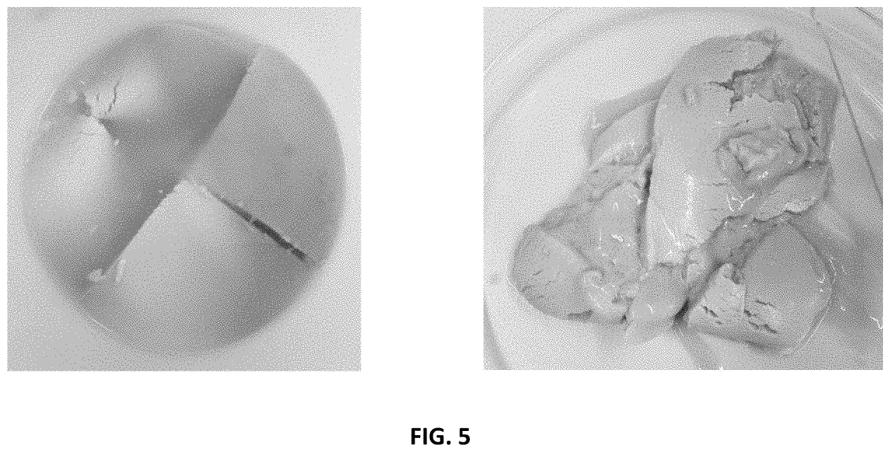

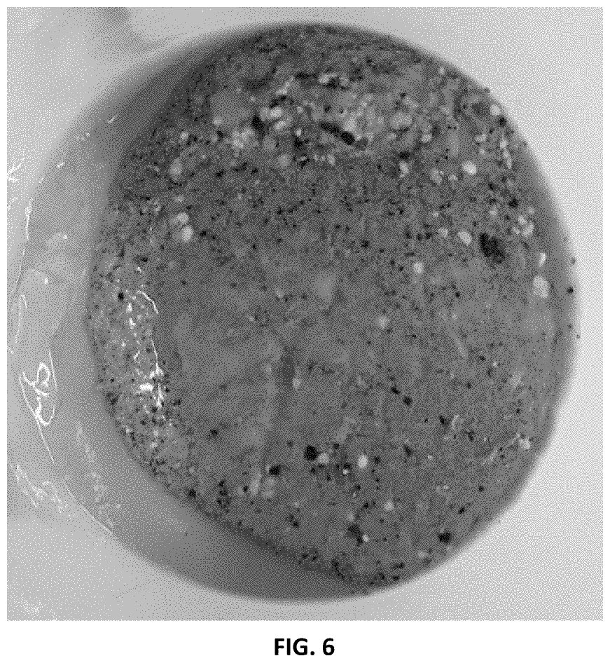

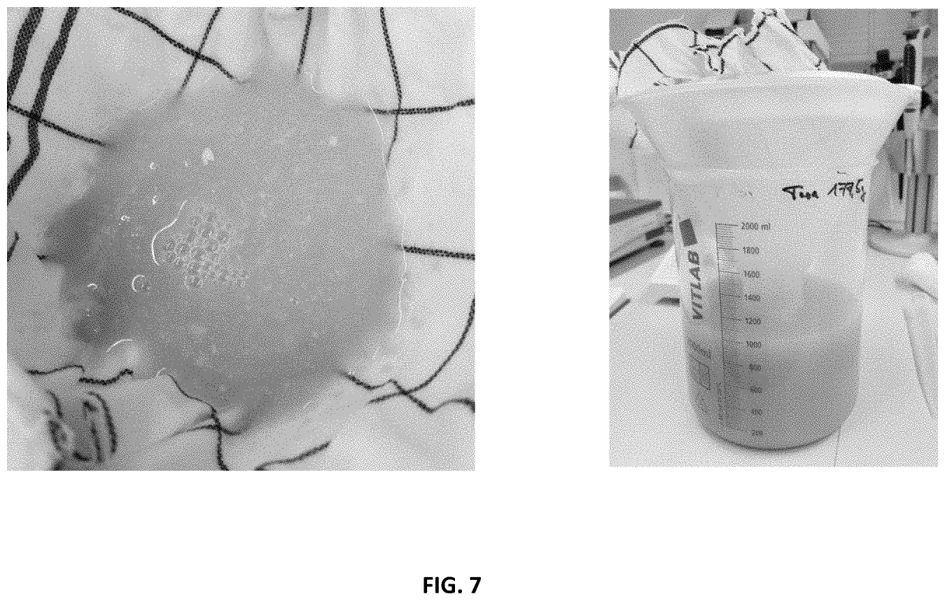

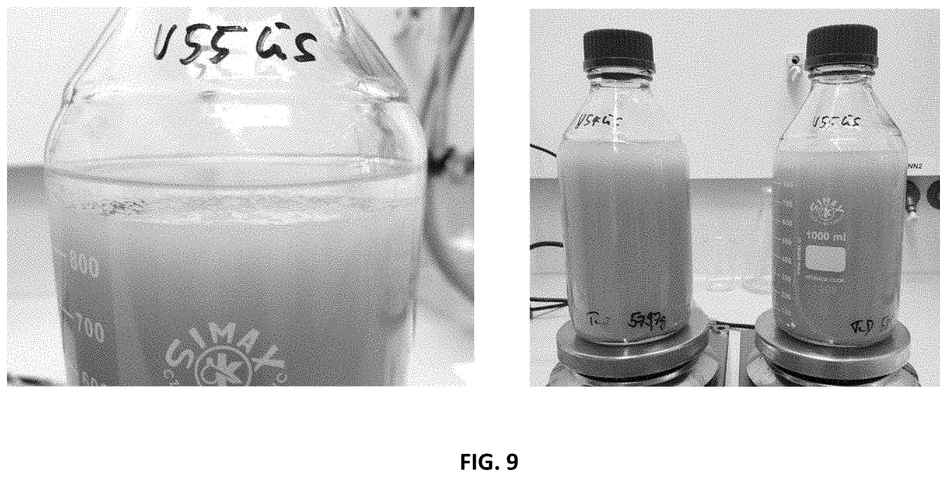

The fat phase (FIG. 5 for each replicate) and solid phase (FIG. 6) were separated from the aqueous phase, and the aqueous phase was further processed by filtration through a cloth filter. FIG. 7 shows solids removed by cloth filtration (left panel) and the resulting filtrate. The filtered aqueous phase was then mixed with a 5% (V/W) of heptane and the resulting solution was stirred overnight at room temperature to defat the solution. FIG. 9, left panel, shows the aqueous phase with heptane added without stirring. FIG. 9, right panel, shows the aqueous phase for each replicate with heptane and with stirring.

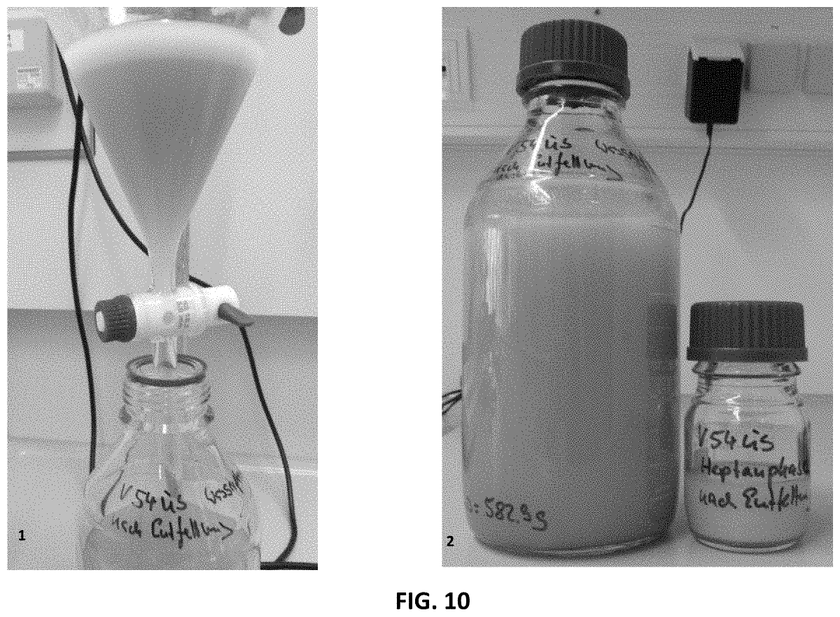

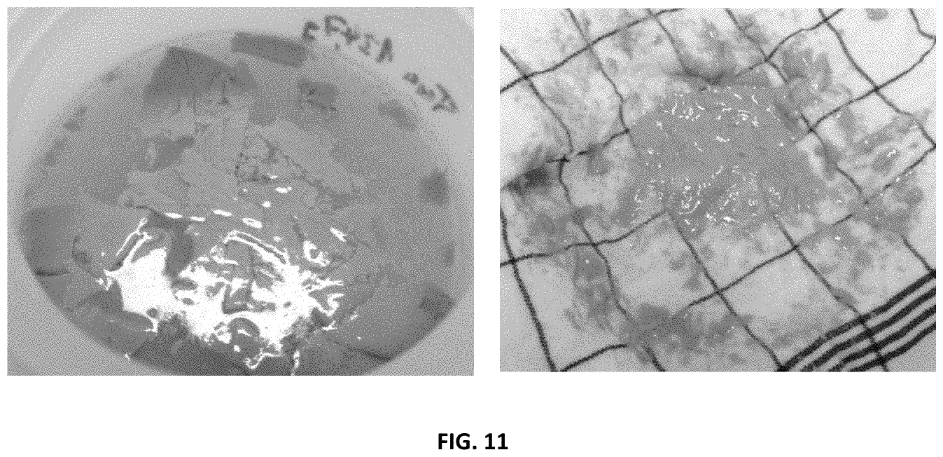

After defatting, the organic phase (heptane containing phase) and the aqueous phase were separated using a separation funnel (FIG. 10, left panel). The separated aqueous phase and organic phase for one of the replicates are shown in FIG. 10, right panel. The defatted and separated aqueous phase was then centrifuged to remove remaining fat (FIG. 11, left panel) and filtered through a cloth filter. FIG. 11, right panel, shows the fat layer removed after filtration.

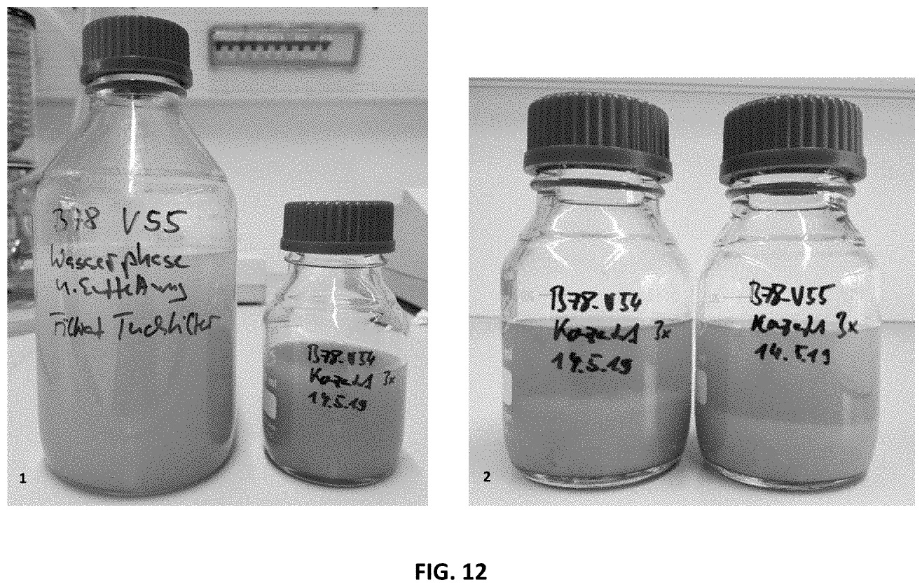

The filtered aqueous phase was then vacuum concentrated in the rotary evaporator at 60° C. until ⅓ of the initial volume was reached. Then the concentrated aqueous phase was incubated at 4° C. overnight. FIG. 12, left panel, shows a filtered aqueous phase prior to concentration (left bottle) and after concentration (right bottle). FIG. 12, right panel, shows the aqueous phase for each replicate after overnight incubation at 4° C.

The efficiency of using acetone or ethanol as precipitation solvent was initially studied. Two precipitation pre-tests were performed, using ethanol or acetone at a ratio of 80% (V/V). Precipitation efficiency was monitored by PAS determination in the concentrate and supernatant.

TABLE 1
Precipitation efficiency obtained from each solvent pre-test.

| | | | Precipitation |
|---|---|---|---|
| Pre-test | Solvent used | Concentration | Efficiency |
| 1 | Ethanol | 80% (v/v) | 93.9% |
| 2 | Acetone | 80% (v/v) | 84.3% |

Due to the better efficiency and the lower toxicity of ethanol, it was selected to perform the precipitation steps in the process.

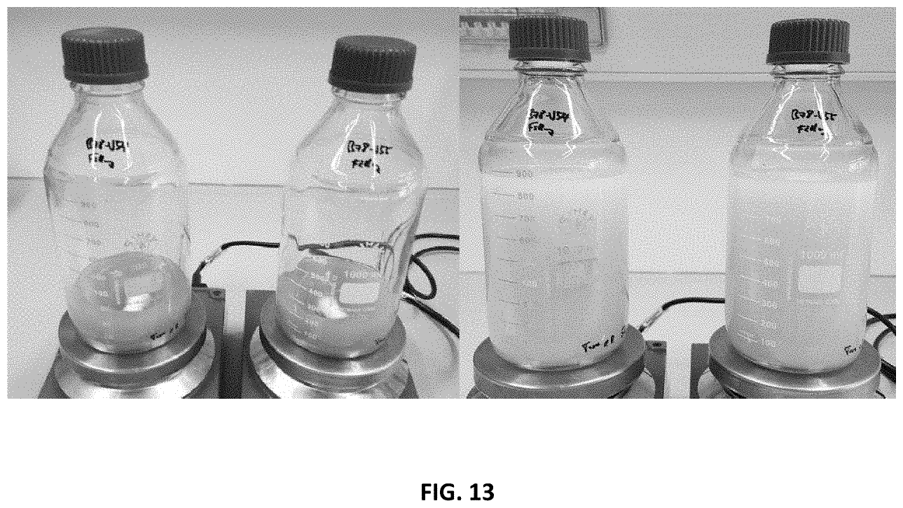

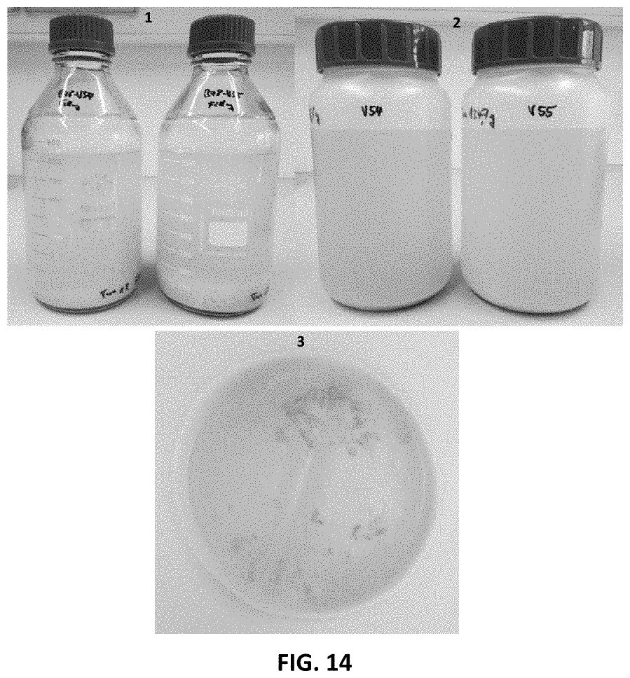

Once the solvent was chosen, precipitation of the concentrated water phase was performed using ethanol at a ratio of 80% (v/v) under stirring at 4° C. FIG. 13, two bottles on the left, shows concentrated aqueous phase without added ethanol for each replicate. FIG. 13, two bottles on the right, show ethanol added to each replicate. Precipitants from the aqueous phase were then isolated by centrifugation at 3500 rpm for 20 minutes at 4° C. FIG. 14, top left two bottles show sediment precipitated in bottles. FIG. 14, top right two bottles show aqueous phase after centrifugation with precipitant pelleted in the bottom. FIG. 14, bottom panel, shows the precipitated substances with the aqueous supernatant removed.

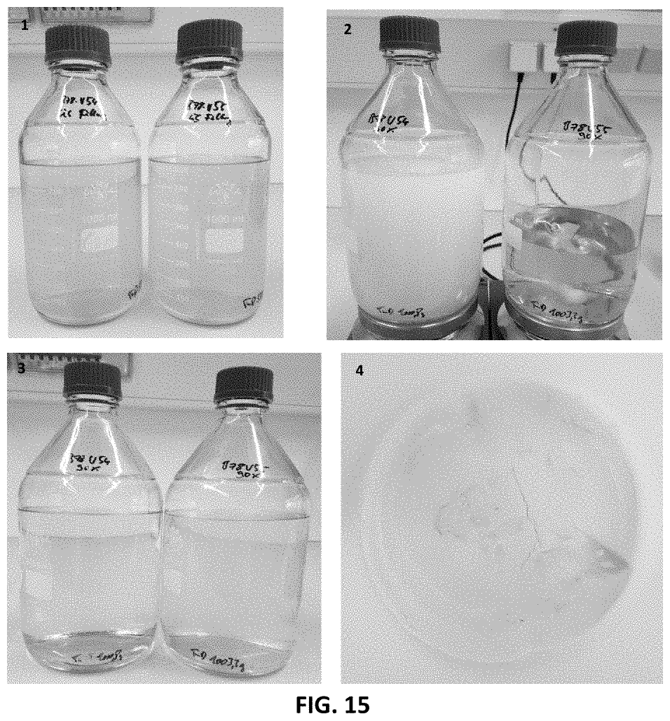

The removed supernatant was turbid and yellowish in color (FIG. 15, top left panel. Thus, another round of precipitation at 90% ethanol (v/v) was performed (FIG. 15, top left panel) followed by centrifugation and separation of the supernatant aqueous phase and the precipitated materials. FIG. 15, bottom left panel, shows that the resulting supernatant is nearly colorless and clear while FIG. 15, bottom right panel, shows recovered precipitate. Based on these results, it was concluded that 90% ethanol is the optimum solvent condition for the precipitation.

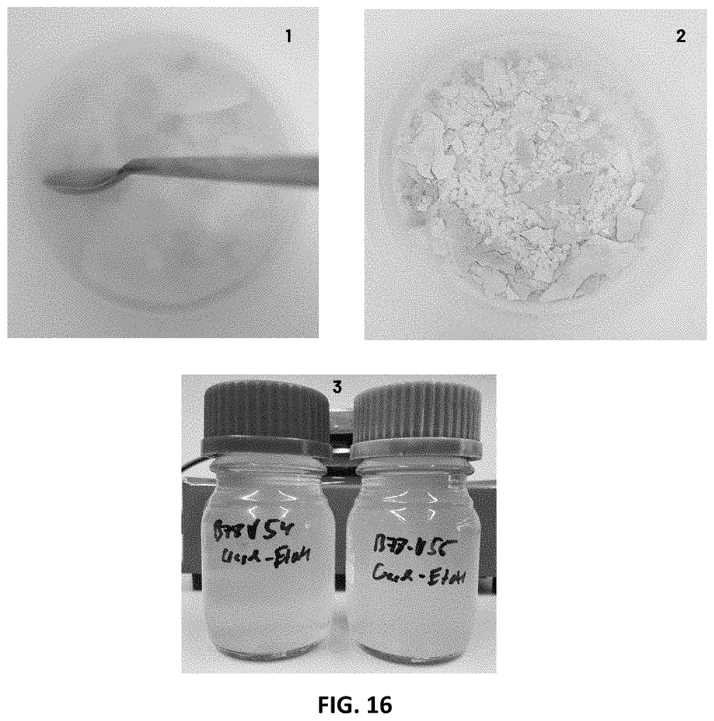

The recovered precipitates were dewatered via two different washing protocols. In the first protocol, 100% ethanol was added with stirring to the precipitate at room temperature. The mixture was centrifuged to allow a better separation of the solid from the supernatant. As supernatant showed some precipitates in suspension, it was decantated overnight and the new precipitate was collected and blended with the first one. See FIG. 16—Left top panel shows precipitate in 100% ethanol, top right panel shows precipitate after recovery via centrifugation and decantation, bottom panel shows cloudy supernatant collected after centrifugation but prior to decantation.

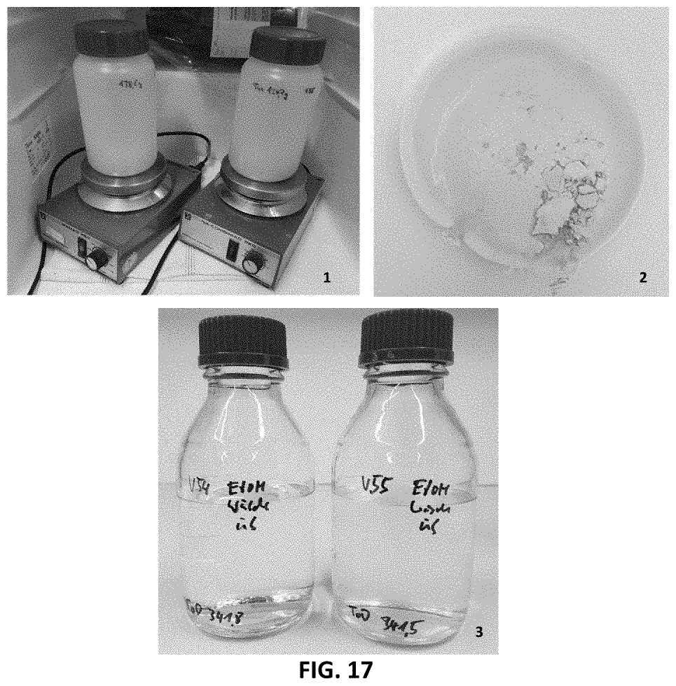

In the second alternate dewatering protocol, 80% ethanol solution was added with stirring at 4° C. overnight. Due to the long stirring time, a finer precipitate was obtained. The solid was isolated by centrifugation to allow a better separation from the supernatant. The supernatant was transparent. See FIG. 17—Top left panel shows precipitate in 80% ethanol with stirring, top right panel shows recovered precipitate after centrifugation and removal of supernatant, bottom panel shows recovered supernatant.

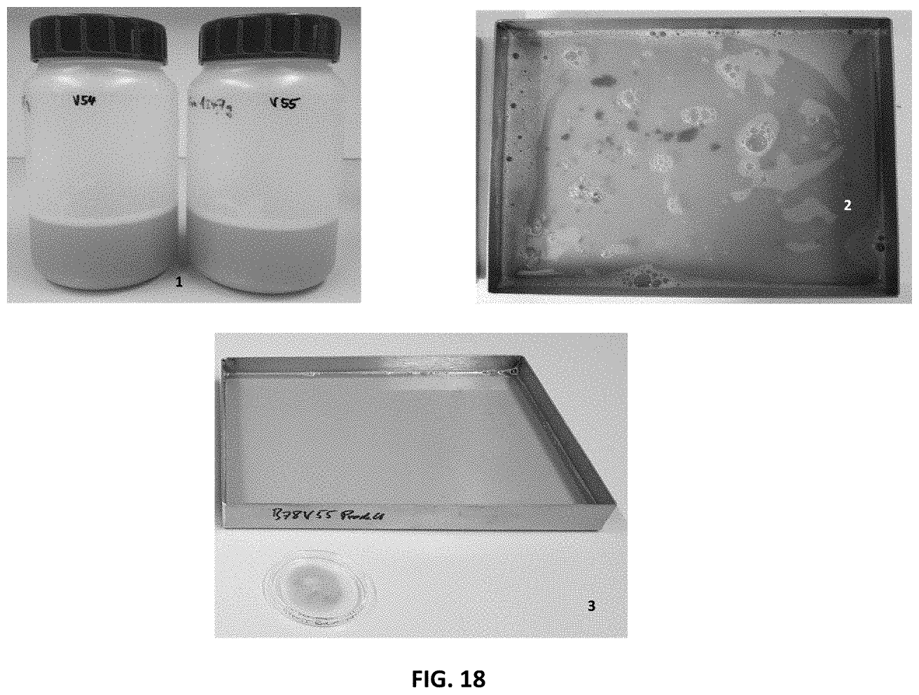

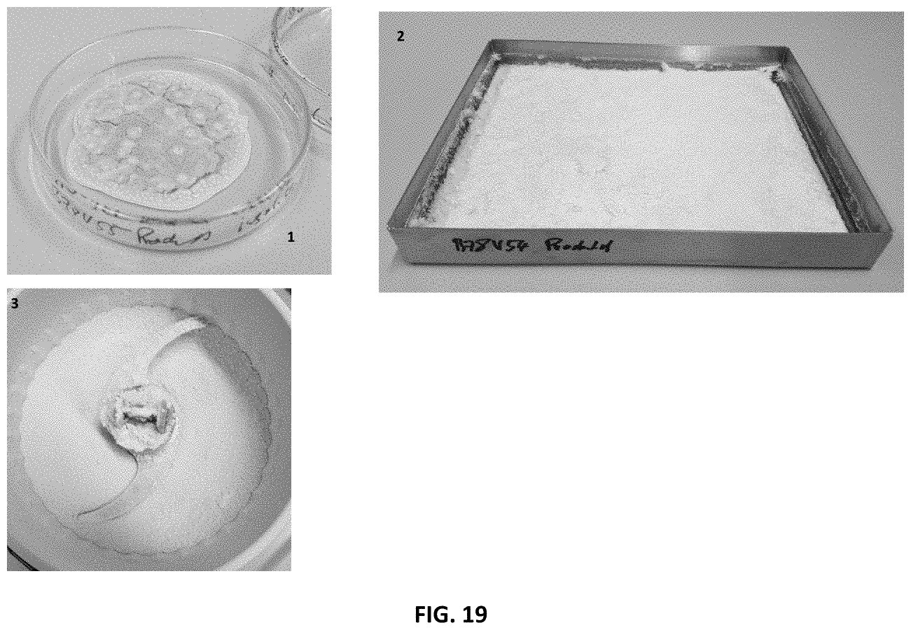

The precipitate was dissolved in ultrapure water, frozen and freeze dried. The freeze-dried sample was then milled. See FIG. 18—Top left panel shows precipitate in ultrapure water, top right panel shows dissolved sample including some insoluble particles, bottom panel shows dissolved sample with insoluble particles removed into a Petri dish. See also FIG. 19—Top left panel showing insoluble particles, top right panel showing dried sample, bottom pane 1 shows the final milled product sometimes referred to herein as GBX102.

The terms of yield the results are the following (Table 1):

| | B78-V54 | B78-V55 | |
|---|---|---|---|
| | (1^{ST} | (2^{ND} | |
| | REPLICATE) | REPLICATE) | MEAN |
| DRY PRODUCT | 3.2 | 3.3 | 3.3 |
| PER 600 G | | | |
| MUCUS AFTER | | | |
| SAMPLING | | | |
| LOSSES (G) | | | |
| DRY PRODUCT | 6.5 | 6.6 | 6.6 |
| PER 600 G | | | |
| MUCUS | | | |
| CORRECTED (G) | | | |
| DRY PRODUCT | 10.9 | 11.0 | 11.0 |
| PER KG MUCUS | | | |
| CORRECTED (G) | | | |
| GLYCAN | 80.6 | 76.7 | 78.7 |
| CONTENT IN | | | |
| THE DRY | | | |
| PRODUCT-PAS | | | |
| TEST (%) | | | |
| GLYCAN PER | 8.79 | 8.44 | 8.6 |
| KG MUCUS- | | | |
| TEST(G) | | | |
| GLYCAN YIELD- | 0.88 | 0.84 | 0.86 |
| PAS TEST (%) | | | |

### Example 2 Bacterial Growth in GBX102 Supplemented Media

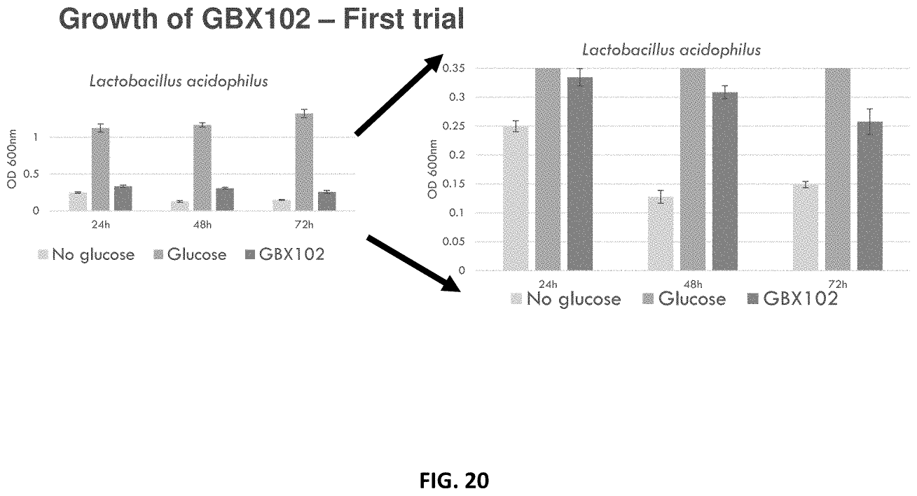

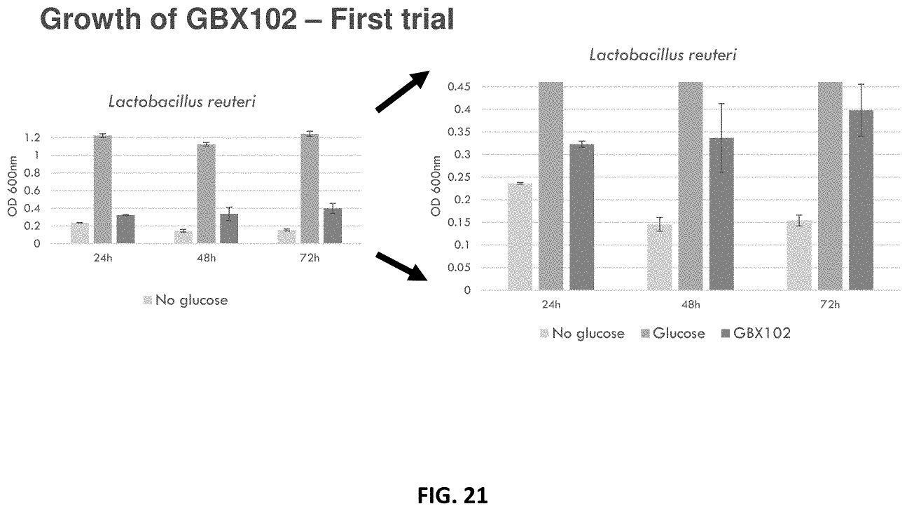

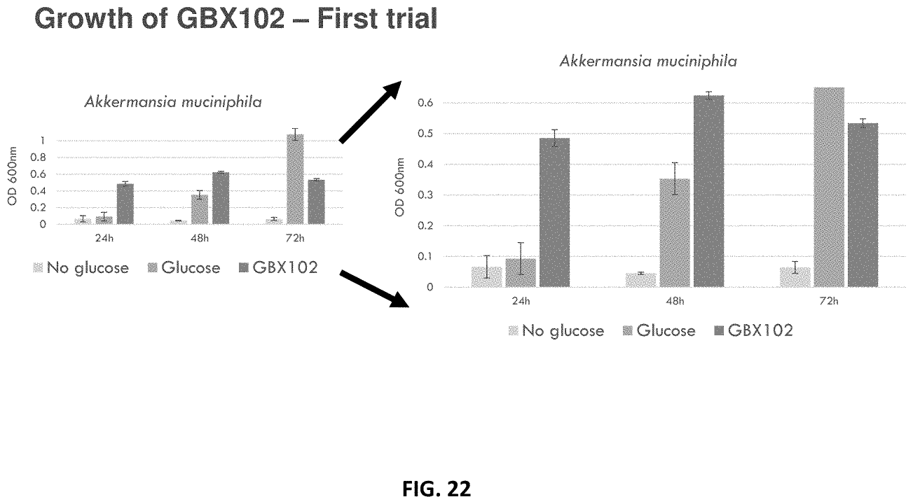

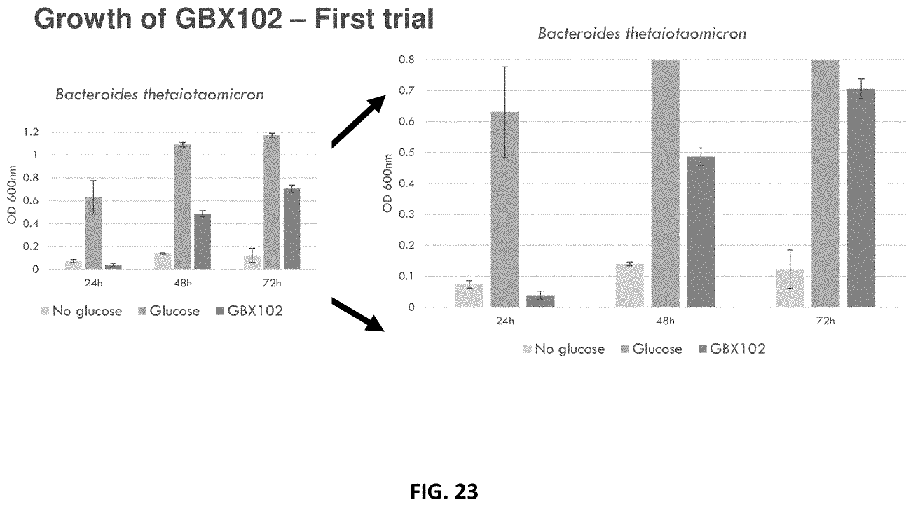

Bacterial growth in the presence of a composition of a claimed invention in liquid minimal media, GBX102 (15 mg/ml), was compared to bacteria growth in liquid minimal media (no glucose) and liquid minimal media with glucose (glucose). GBX102 in the form of a dried powder was obtained by the process of Example 1. Each sample was added to 200 μl medium and inoculated with 5 μl of Lactobacillus acidophilus (FIG. 20), Lactobacillus reuteri (FIG. 21), Akkermansia muciniphila (FIG. 22), or Bacteroides thetaiotaomicron (FIG. 23). Each sample was prepared in triplicate. The bacterial growth was determined by measuring the optical densities (OD) at 600 nm in a spectrophotometer after 24 h, 48 h, and optionally 72 h of growth starting with an OD of 0.05.

FIG. 20 illustrates that supplementing minimal media with GBX102 results in growth of Lactobacillus acidophilus, as measured by OD, superior to growth of Lactobacillus acidophilus in no glucose at 24, 48, and 72 hours. It is believed that glucose is not an ideal energy source for gut microbiota, as glucose tends to inhibit the growth of certain beneficial bacteria in the microbiota, such as Akkermansia muciniphila.

FIG. 21 illustrates that supplementing minimal media with GBX102 results in growth of Lactobacillus reuteri, as measured by OD, superior to growth of Lactobacillus reuteri in no glucose at 24, 48, and 72 hours.

FIG. 22 illustrates that supplementing minimal media with GBX102 results in growth of Akkermansia muciniphila, as measured by OD, superior to growth of Akkermansia muciniphila in no glucose at 48 and 72 hrs. Significantly, GBX102 supplementation in minimal media also resulted in higher Akkermansia muciniphila growth than glucose supplementation at 48 hrs.

FIG. 23 illustrates that supplementing minimal media with GBX102 results in growth of Bacteroides thetaiotaomicron, as measured by OD, superior to growth of Bacteroides thetaiotaomicron in no glucose at 48 and 96 hours.

The results shown in FIGS. 20-23 show that compositions of the claimed invention sustain higher growth rates for some beneficial bacteria at different time points that minimal media. Thus, these results suggest that beneficial bacteria are capable of utilizing glycans attached to peptides or proteins, especially after other energy sources are exhausted.

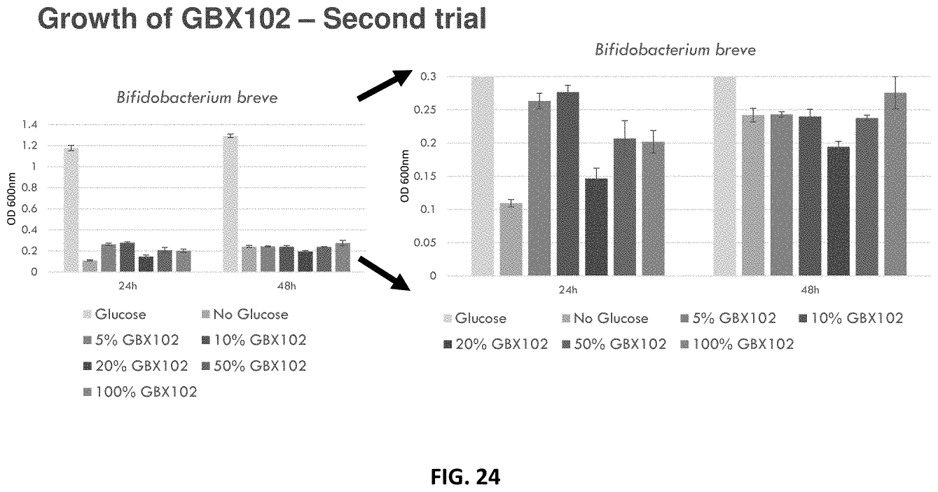

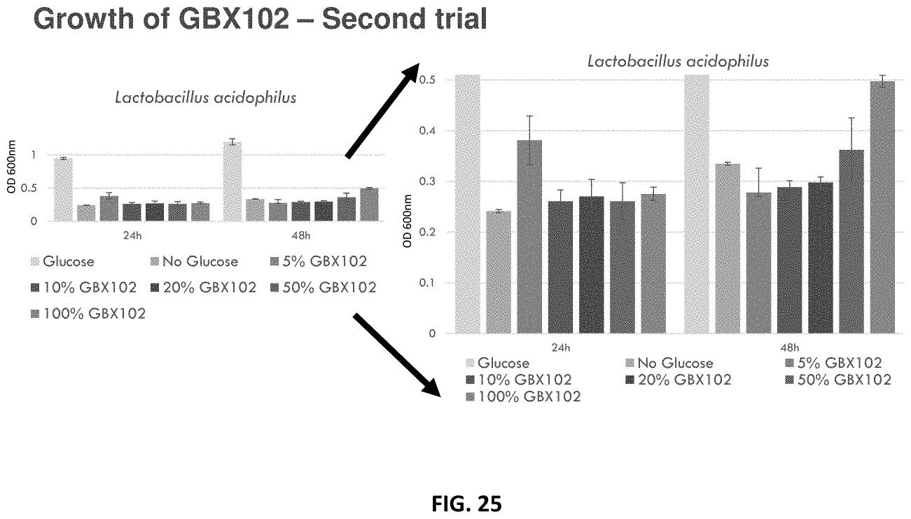

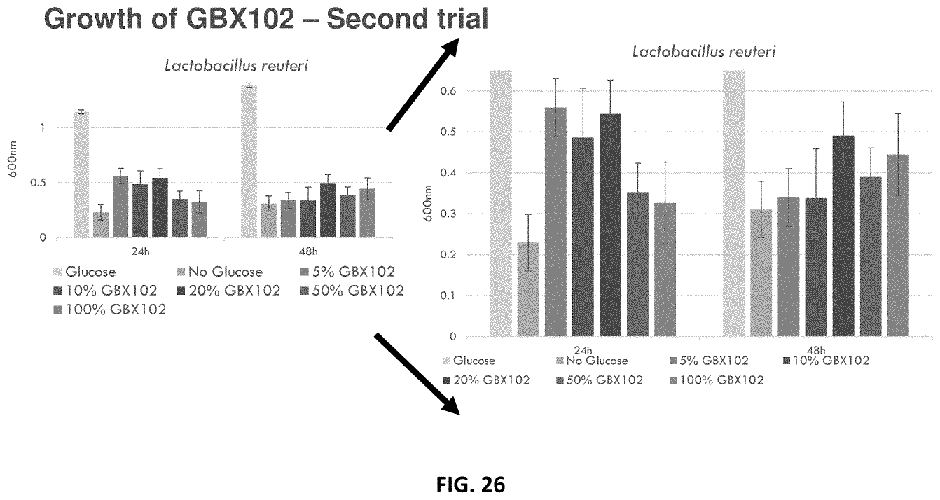

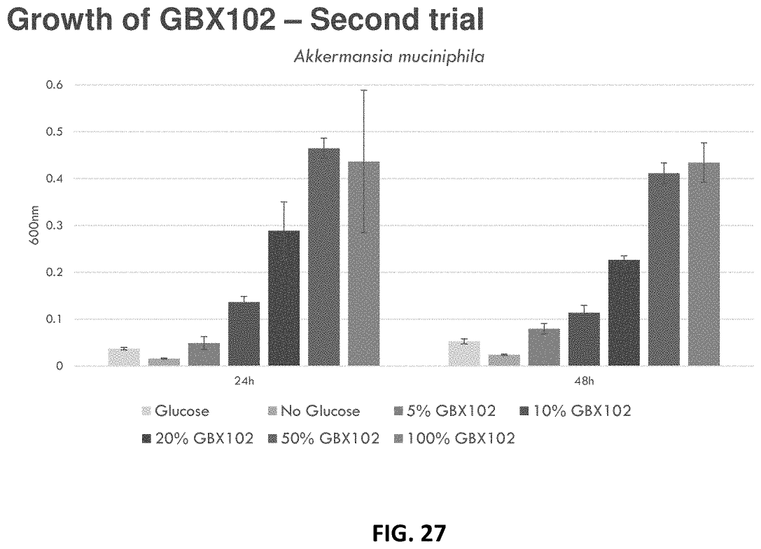

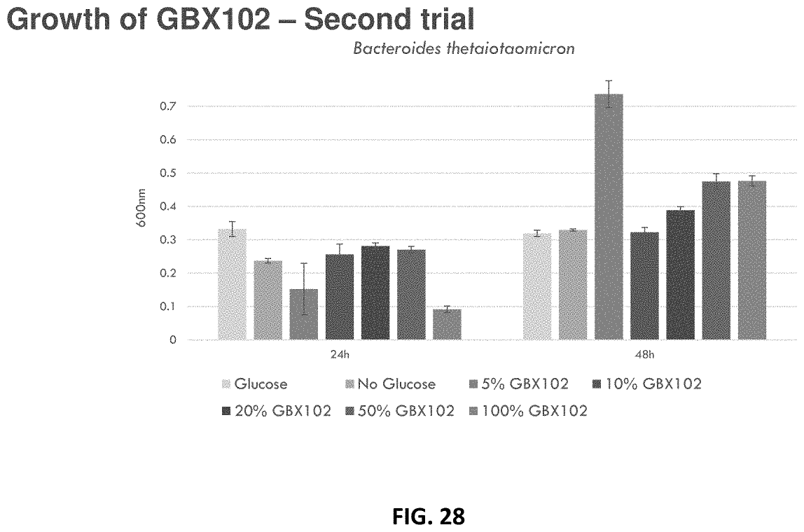

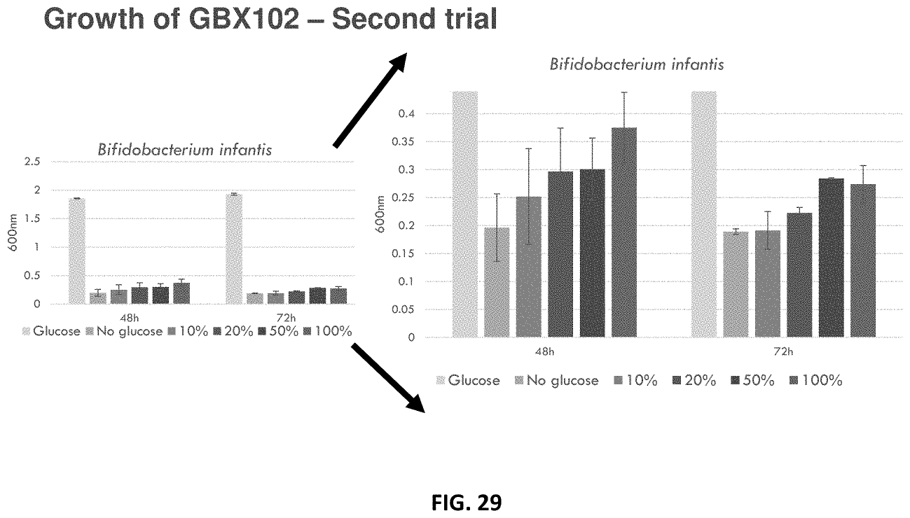

Bacterial growth in the presence of glucose and GBX102 at different concentrations (starting with 15 mg/ml as 100% and diluting 50% (i.e., 7.5 mg/ml), 20%, 10% and 5%) was compared to bacteria growth in liquid minimal media (no glucose) and liquid minimal media with glucose (glucose). GBX102 in the form of a dried powder was obtained by the process of Example 1. Each sample was added to 200 μl medium and inoculated with 5 μl of Bifidobacterium breve (FIG. 24), Lactobacillus acidophilus (FIG. 25), Lactobacillus reuteri (FIG. 26), Akkermansia muciniphila (FIG. 27), Bacteroides thetaiotaomicron (FIG. 28), or Bifidobacterium infantis (FIG. 29). Each sample was prepared in triplicate. The bacterial growth was determined by measuring the optical densities (OD) at 600 nm in a spectrophotometer after 24 h and 48 h of growth starting with an OD of 0.05.

FIG. 24 illustrates that supplementing minimal media with GBX102 results in growth of Bifidobacterium breve, as measured by OD, superior to growth of Bifidobacterium breve in no glucose at 24 and 48 hr.

FIG. 25 illustrates that supplementing minimal media with GBX102 results in growth of Lactobacillus acidophilus, as measured by OD, superior to growth of Lactobacillus acidophilus in no glucose at 24 and 48 hours.

FIG. 26 illustrates that supplementing minimal media with some concentrations of GBX102 results in growth of Lactobacillus reuteri, as measured by OD, superior to growth of Lactobacillus reuteri in no glucose at 24 and 48 hrs.

FIG. 27 illustrates that supplementing minimal media with GBX102 results in growth of Akkermansia muciniphila, as measured by OD, superior to growth of Akkermansia muciniphila in no glucose at 24 and 48 hours. Significantly, Akkermansia muciniphila growth was dependent on GBX102 concentration.

FIG. 28 illustrates that supplementing minimal media with some concentrations of GBX102 results in growth of Bacteroides thetaiotaomicron, as measured by OD, superior to growth of Bacteroides thetaiotaomicron in no glucose at 48 hours.

FIG. 29 illustrates that supplementing minimal media with some concentrations of GBX102 results in growth of Bifidobacterium infantis, as measured by OD, superior to growth of Bifidobacterium infantis in no glucose at 48 and 72 hours.

The results shown in FIGS. 24-29 show that compositions of the claimed invention can sustain higher growth rates for some beneficial bacteria at different time points that minimal media. Thus, these results suggest that beneficial bacteria are capable of utilizing glycans attached to peptides or proteins, especially after other energy sources are exhausted. In particular, the results shown in FIGS. 27 and 29 show that at some time points, growth of Akkermansia muciniphila and Bifidobacterium infantis was dependent upon GBX102 dosage.

### Example 3

Project: Conjugated glycans to improve outcomes in immuno-oncology therapies and inflammation.

Background

Gnubiotics has developed a pipeline of conjugated glycan-based candidates (e.g., GBX102) that can modulate the microbiome, activate metabolites and exert antimicrobial as well as immunomodulatory effects. Those effects should result in protection and maturation of intestinal epithelial barrier, protection against pathogens and restoration of a well-balanced immune system.

Based on the proposed mode-of-action, Gnubiotics glycans could be highly attractive oral therapeutic agents for the treatment of dysbiotic diseases. In particular, they could be used as anti-cancer drugs, to enhance the efficacy of anti-cancer drugs, such as ICI inhibitors, ameliorated side effects induced by anti-cancer drugs, such as ICIs, or act as anti-inflammatory agents to treat chronic inflammatory diseases, such as inflammatory bowel disease (IBD).

Approach

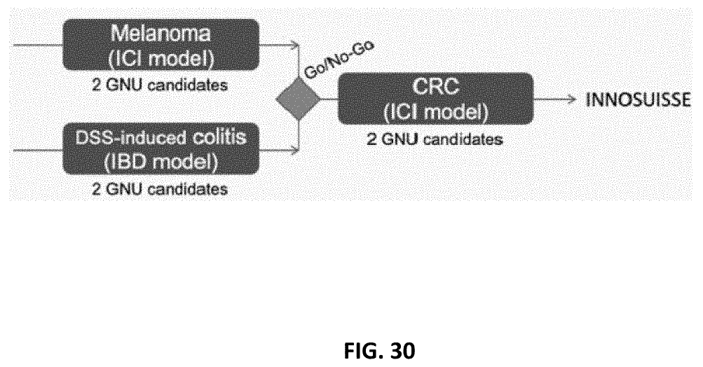

The program follows a step-wise approach to mitigate risks. Each experimental block is terminated by a Go/No-go gate consisting in a review meeting to inform next steps (FIG. 30).

BLOCK 1: MELANOMA (ICI MODEL). The first experimental block studies the efficacy of Gnubiotics candidates (e.g., GBX102) in subcutaneous melanoma mouse model in absence/presence of ICI treatment.

BLOCK 2: DSS-INDUCED COLITIS (IBD MODEL). The second experimental block studies the efficacy of Gnubiotics candidates (e.g., GBX102) in DSS-induced acute colitis mouse model.

BLOCK 3: CRC (ICI MODEL). The third experimental block studies the efficacy of Gnubiotics candidates in subcutaneous colorectal cancer (CRC) mouse model in absence/presence of ICI treatment.

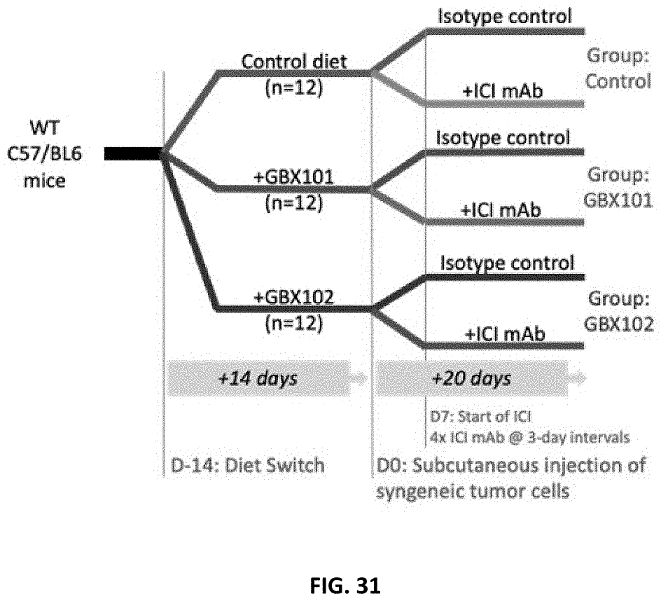

Block 1: Melanoma (ICI Model)

| Objectives | Primary: | | | |
|---|---|---|---|---|
| | Demonstrate that experimental products (e.g., GBX102) reduce tumor | | | |
| | size when administered alone or in combination with ICIs vs control | | | |
| | group. | | | |
| | Secondary: | | | |
| | Evaluation of the impact of the treatment on (i) immune cell populations | | | |
| | infiltrating the tumor tissue, (ii) inflammatory markers and metabolites | | | |
| | & (iii) gut microbial diversity & composition. | | | |
| PI & Facility | Prof. Scharl's laboratory | | | |
| | Translational Microbiome Research Center, University Hospital Zurich, | | | |
| | University of Zurich | | | |
| Animal model | Mouse strain: Wild-type C57/BL6 (female, 6-8 wks old) | | | |
| | Heterotopic model: Subcutaneous injection using either YUMM1.5 | | | |
| | (preferred option) or B16 (in case YUMM1.5 not easily accessible) | | | |
| | melanoma tumor cells | | | |
| Experimental | Experimental products are mixture of purified mucin-derived O-glycans | | | |
| products | (e.g., GBX102). The products are natural source of complex & diverse | | | |
| | conjugated-glycans. Following candidates are to be tested: | | | |
| | | GBX102: LEAD CANDIDATE | | |
| | | GBX101 | | |
| Control & ICI | Vehicle with isotype control & vehicle with ICI | | | |
| | ICI: Nivolumab (anti-PD-1) | | | |
| Dosing regimen & | Experimental products are provided in drinking water at 3% w/v | | | |
| administration | | | | |
| Control Base | Autoclaved chow diet ad libitum (LabDiet 5K67, Purina Foods) | | | |
| Diet | | | | |
| Study Duration | Total study duration: 34 days | | | |
| | | Acclimation phase to diet: D −14 to D 0 | | |
| | | Tumor injection: D 0 | | |
| | | ICI treatment start: D 7 | | |
| | | Final read-out: D 20 (note: tumor volumes will be monitored | | |
| | | closely throughout the study and in case of obvious difference | | |
| | | between treatment arms on D 15, all mice will be euthanized on | | |
| | | D 15) | | |
| | For details of study intervention and assessment, see Table “STUDY | | | |
| | INTERVENTION & ASSESSMENT”. | | | |
| Sample size and | See FIG. 31 | | | |
| study arms | | | | |
| | | END-POINT | DESCRIPTION | TIMEPOINT |
| End-points | | Tumor volume | PRIMARY EP: To | Twice a week, |
| | | | assess impact on tumor | starting on D 0 |
| | | | evolution | |
| | | Tumor weights | PRIMARY EP: To | D 20 (end of exp) |
| | | | assess impact on tumor | |
| | | | evolution | |
| | | TILs & subsets | To characterize anti- | D 20 (end of exp) |
| | | (from tumor | tumor immunity | |
| | | sample) | HOW: | |
| | | | Scharl's panels: innate & | |
| | | | adaptive, ICI, Treg, Th, | |
| | | | CD4/CD8, MHC | |
| | | | Example of immune | |
| | | | cells: | |
| | | | Total & effector CD4 + | |
| | | | subsets (e.g. IEN-γ+, | |
| | | | Foxp3+, Th), Total & | |
| | | | effector CD8 + subsets, | |
| | | | Total DCs + subsets (e.g. | |
| | | | pDCs), MHC class | |
| | | | expression on DC, B | |
| | | | cells, Macrophages, NK | |
| | | | cells | |
| | | 16S | To assess microbiome | D 0 |
| | | (from fecal | modulation activity & | D 20 (end of |
| | | samples) | link to anti-tumor | exp) |
| | | | immunity | OPTIONAL: |
| | | | | D −14 |
| | | | | (baseline) |
| | | Cytokine panel | To characterize systemic | D 0 |
| | | (from serum) | inflammation-promoting | D 20 |
| | | | effect | |
| | | | HOW: | |
| | | | Bio-Plex Pro ™ Mouse | |
| | | | Cytokine Group I Panel | |
| | | | 23-plex (BioRad) | |
| | | Untargeted | e.g. to look for inosine | D 0 |
| | | metabolomics | | D 20 |
| | | (from serum) | | |
| | Remarks: | | | |
| | The measurements for secondary end-points (TILs, 16S, Cytokine panel, | | | |
| | untargeted metabolomics) is executed after completion of an initial | | | |
| | analysis of the primary end-points to guide the selection of the groups. | | | |
| | | TYPE | RATIONAL | TIMEPOINT |
| Additional | | Cecum & colon | To enable the | D 20 |
| specimen | | | complementary | |
| collection | | | following analysis: | |
| (no upfront | | | 16S, SCFA, MPO, | |
| analysis) | | | FACS of immune | |
| | | | cells, cytokine panel | |
| | | Colon tissue | To enable the | D 20 |
| | | | complementary | |
| | | | following analysis: | |
| | | | HE, Acian blue, | |
| | | | IHC, length | |
| | | | assessment | |
| | | Additional stool | To enable the | D 20 |
| | | | complementary | |
| | | | following analysis: | |
| | | | SCFA | |

TABLE
STUDY INTERVENTION & ASSESSMENT

| | | | |
|---|---|---|---|
| D −14 | D 0 | D 7 | D 20 |

MAJOR STUDY EVENTS

| | | | | |
|---|---|---|---|---|
| Randomization | X | | | |
| (to different diet | | | | |
| groups) | | | | |
| Tumor injection | | X | | |
| ICI treatment start | | | X | |
| Study end | | | | X |

COLLECTION

| | | | |
|---|---|---|---|
| Tumor | | | X |
| Serum | | X | X |
| Stool | X | X | X |
| Cecum & colon | | | X |
| content | | | |
| Colon tissue | | | X |

END-POINTS

| Tumor volume | | TWICE A WEEK | |
|---|---|---|---|
| Tumor weights | | | X |
| TILS | | | X |
| 16S | (OPTIONAL) | X | X |
| Cytokine panel | | X | X |
| Untargeted | | X | X |
| metabolomic | | | |

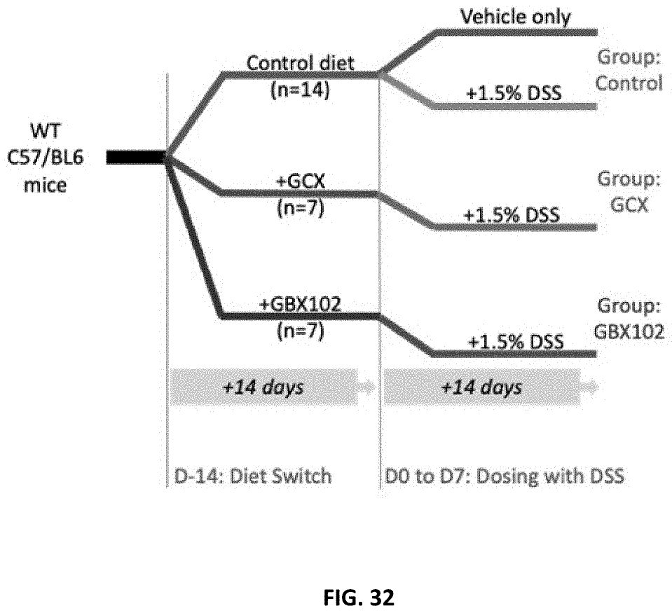

Block 2: DSS-Induced Acute Colitis (IBD Model)

| Objectives | Primary: | | | |
|---|---|---|---|---|
| | Demonstrate that experimental products reduce disease activity index | | | |
| | (DAI) of acute colitis when administered vs control group. | | | |
| | Secondary: | | | |
| | Evaluation of the impact of the treatment on (i) immune cells population | | | |
| | in the intestine, (ii) inflammatory markers & (iii) gut microbial diversity & | | | |
| | composition. | | | |
| PI & Facility | Prof. Scharl's laboratory | | | |
| | Translational Microbiome Research Center, University Hospital Zurich, | | | |
| | University of Zurich | | | |
| Animal model | Mouse strain: Wild-type C57/BL6 (female, 6-8 wks old) | | | |
| | DBS model: 1.5% DSS treatment for 7 days (in the drinking water) | | | |
| | induces barrier damage and immune cell infiltration. | | | |
| | Rational: Experimental products will restore epithelial barrier, ameliorate | | | |
| | intestinal dysbiosis & exert immunoregulatory function. | | | |
| Experimental | Experimental products are mixture of purified mucin-derived O-glycans. | | | |
| products | The products are natural source of complex & diverse conjugated-glycans. | | | |
| | Following candidates are expected to be tested: | | | |
| | | GBX102: LEAD CANDIDATE | | |
| | | GCX | | |
| Control | Vehicle | | | |
| Dosing regimen | Experimental products are provided in drinking water at 1-3% w/v | | | |
| & | | | | |
| administration | | | | |
| Control Base | Autoclaved chow diet ad libitum (LabDiet 5K67, Purina Foods) | | | |
| Diet | | | | |
| Study Duration | Total study duration: 28 days | | | |
| | | Acclimatation phase to diet: D −14 to D 0 | | |
| | | Induction of colitis with DSS: D 0 | | |
| | | Stop of DSS: D 7 | | |
| | | Final read-out: D 14 | | |
| | For details of study intervention and assessment, see Table “STUDY | | | |
| | INTERVENTION & ASSESSMENT”. | | | |
| Sample size and | A total of 28 mice will be randomized in 4 arms (see, FIG. 32) | | | |
| study arms | | | | |
| | | END-POINT | DESCRIPTION | TIMEPOINT |
| End-points | | Disease activity | PRIMARY EP: To assess impact | Daily, starting |
| | | index (DAI) | on colitis. | on D 0 |
| | | | HOW: | |
| | | | BW, presence of blood and stool | |
| | | | consistency | |
| | | Endoscopy | PRIMARY EP: To assess impact | D 14 (end of |
| | | | on colitis. | exp) |
| | | HE & alcian | PRIMARY EP: HE to assess | D 14 (end of |
| | | blue of colon | impact on colitis (severity of | exp) |
| | | | tissue damage) & AB to | |
| | | | quantitate acidic glycans on GI | |
| | | | tract and thus mucus secretion. | |
| | | Macroscopic | To assess inflammation status | D 14 (end of |
| | | markers of | HOW: | exp) |
| | | inflammation | Colon length & spleen weight | |
| | | IHC of colon | To assess gut barrier integrity. | D 14 (end of |
| | | | HOW: | exp) |
| | | | Tight-junction proteins: ZO-1, | |
| | | | Occludin, Claudin-1 | |
| | | MPO assay | To measure extent of | D 14 (end of |
| | | (from colon) | inflammatory cell invasion in the | exp) |
| | | | colon | |
| | | Immune cells | To assess immune status. | D 14 (end of |
| | | (from intestine) | HOW: | exp) |
| | | | Scharl's panels: innate & | |
| | | | adaptive, ICI, Treg, Th, | |
| | | | CD4/CD8, MHC | |
| | | 16S | To assess microbiome | D 0 |
| | | (from fecal | modulation activity. | D 14 (end of |
| | | samples) | | exp) |
| | | | | OPTIONAL: |
| | | | | D −14 (baseline) |
| | | Cytokine panel | To characterize | D 0 |
| | | (from serum) | immunomodulatory and anti- | D 14 |
| | | | inflammatory effects. | |
| | | | HOW: | |
| | | | Bio-Plex Pro ™ Mouse Cytokine | |
| | | | Group I Panel 23-plex (BioRad) | |
| | Remarks: | | | |
| | The measurements for secondary end-points (IHC of the colon, MPO | | | |
| | assay, Immune cell populations, 16S, cytokine panel) will only be | | | |
| | executed after completion of an initial analysis of the primary end-points | | | |
| | to guide the selection of the groups that will be analyzed. | | | |
| | | TYPE | RATIONAL | TIMEPOINT |
| Additional | | Additional cecum & | To enable the | D 14 |
| specimen | | colon | complementary | |
| collection | | | following analysis: | |
| (no upfront | | | 16S, SCFA, cytokine | |
| analysis) | | | panel | |
| | | Additional stool | To enable the | D 14 |
| | | | complementary | |
| | | | following analysis: | |
| | | | SCFA | |

TABLE
STUDY INTERVENTION & ASSESSMENT

| | | | |
|---|---|---|---|
| D −14 | D 0 | D 7 | D 14 |

MAJOR STUDY EVENTS

| Randomization | X | | |
|---|---|---|---|
| (to different diet | | | |
| groups) | | | |
| DSS injection | | X (D 0-D 7) | |
| Study end | | | X |

COLLECTION

| | | | |
|---|---|---|---|
| Cecum & colon | | | X |
| content | | | |
| Colon tissue | | | X |
| Spleen | | | X |
| Serum | | X | X |
| Stool | X | X | X |

END-POINTS

| DAI | | DAILY | |
|---|---|---|---|
| (BW & Stool | | | |
| observation) | | | |
| Endoscopy | | | X |
| Macroscopic | | | X |
| markers of | | | |
| inflammation | | | |
| (colon length & | | | |
| spleen weight) | | | |
| HE & AB of colon | | | X |
| IHC of colon | | | X |
| MPO | | | X |
| Immune cells | | | X |
| 16S | (OPTIONAL) | X | X |
| Cytokine panel | | X | X |

Block 3: CRC (ICI Model)

Same study design as block 1, but with MC38 tumor cells instead of melanoma cells.

What is claimed is:

1. A composition comprising a mixture of glycoproteins obtained from mucins of the outer mucus layer of pig stomach, wherein:
a) the composition is obtained without subjecting the mucins to conditions or reagents that release oligosaccharides from glycoproteins and glycopeptides;
b) glycoprotein content of the composition is greater than about 70% (w/w); and
c) the free glycan content of the composition is less than 1% (w/w).

2. The composition of claim 1, wherein the oligosaccharide content of the composition is greater than or equal to about 35% (w/w).

3. The composition of claims 1-2, wherein the composition has a salt content of less than about 2%.

4. The composition of claims 1-3, wherein the composition is a powder and has a glycoprotein content of greater than 75% by weight.

5. The composition of claims 1-4, wherein the composition has a free glycan content of less than 0.1% by weight.

6. A nutritional or dietary composition, nutritional or dietary premix, or infant formula comprising a composition according to any one of claims 1-5.

7. An animal feed or animal feed supplement comprising a composition according to any one of claims 1-5.

8. A method of manufacturing a composition comprising a mixture of
glycopeptides, comprising the following steps a)-g):
a) providing a composition comprising mucins from the outer mucus layer of pig stomach or a partially purified fraction thereof and water;
b) adjusting the pH of the composition to 3.0 to 3.5 with the addition of an acid and incubating the solution to hydrolyze the composition;
c) isolating an aqueous phase from the composition;
d) defatting the isolated aqueous phase;
e) precipitating and isolating a composition comprising glycopeptides from the defatted aqueous phase;
f) dewatering the isolated composition; and
g) drying the dewatered composition to obtain a composition comprising a mixture of glycopeptides;
wherein the composition comprising a mixture of glycopeptides has an glycopeptide content of greater than or equal to about 70% (w/w) and has a free glycan content of less than 1% (w/w).

9. The method of claim 8, wherein the composition of step a) has been homogenized.

10. The method of claims 8-9, wherein the composition of step a) comprises about a 1:1 ratio of pig stomach outer mucus layer to water.

11. The method of claims 8-10, wherein the pH is adjusted in step b) with HCl.

12. The method of claims 8-11, wherein the composition is incubated in step b) at a pH of 3.0 to 3.5 for 2-4 hours at 45° C.

13. The method of claims 8-12, wherein step b) further comprises adding 1 part of an aqueous solution having a pH of 3.0 to 3.5 to 2-3 parts of the composition after incubation.

14. The method of claims 8-13, wherein the aqueous phase is isolated in step c) by a process comprising centrifugation followed by removal of the aqueous phase.

15. The method of claims 8-14, wherein the aqueous phase obtained in step c) is filtered to remove insoluble material prior to step d).

16. The method of claims 8-15, wherein the isolated aqueous phase is defatted in step d) by the addition of about 5% v/w heptane followed by incubation for 6-18 hours and removal of the heptane phase.

17. The method of claims 8-16, wherein the defatted aqueous phase is filtered to remove insoluble material prior to step e).

18. The method of claim 8-17, wherein the defatted aqueous phase is concentrated to ½ to ¼ of the initial volume prior to step e).

19. The method of claims 8-18, wherein the composition is precipitated in step e) with ethanol or acetone at about 4° C.

20. The method of claims 8-19, wherein the composition is isolated in step e) by filtration or centrifugation after precipitation.

21. The method of claims 8-20, wherein the composition is dewatered in step f) with ethanol.

22. The method of claims 8-21, wherein drying the dewatered composition of step g) comprises freeze drying or rotary evaporation.

23. The method of claims 8-22, wherein the composition of step b) comprises pepsin.

24. The method of claims 8-23, wherein the composition of step a) has not been subject to conditions or reagents that release oligosaccharides from glycoproteins and glycopeptides.

25. A composition comprising a mixture of glycoproteins obtained by the method of claims 8-24.

26. A method of treating, preventing, or reducing the severity of a pathogenic microorganism infection of the gut of a subject comprising orally administering to the subject the composition of claims 1-7.

27. The method of claim 26, wherein the pathogenic microorganism is selected from Escherichia coli, Helicobacter pylori, Streptococcus spp., Toxoplasma gondii, Plasmodium falciparum, influenza virus, rotavirus, and respirovirus.

28. The method of claim 26, wherein the pathogenic microorganism is Escherichia coli.

29. A method of increasing the growth of commensal bacteria in the gut of a subject comprising orally administering to the subject the composition of claims 1-7.

30. The method of claim 29, wherein the commensal bacteria comprise Lactobacillus acidophilus, Lactobacillus reuteri, Akkermansia muciniphila, Bacteroides thetaiotaomicron, Bifidobacterium breve, or Bifidobacterium infantis.

31. A method of reducing the fat mass of a subject comprising orally administering to the subject the composition of claims 1-7.

32. A method of treating, preventing, or reducing inflammation in a subject comprising orally administering to the subject the composition of claims 1-7.

33. The method of claim 32, wherein administration of the composition reduces a level of calprotectin in the blood stream or stool of the subject.

34. A method of increasing production of short chain fatty acid (SCFA) in the gut of a subject comprising orally administering to the subject the composition of claims 1-7.

35. A method of claim 34, wherein the pH in the gut of the subject is decreased.

36. A method of improving gut barrier integrity in the gut of a subject comprising orally administering to the subject the composition of claims 1-7.

37. A method of treating, preventing, or reducing the severity of a pathogenic microorganism infection of the gut of a subject comprising orally administering to the subject a composition produced by the method of any one of claims 8-24.

38. The method of claim 37, wherein the pathogenic microorganism is selected from Escherichia coli, Helicobacter pylori, Streptococcus spp., Toxoplasma gondii, Plasmodium falciparum, influenza virus, rotavirus, and respirovirus.

39. A method of reducing the fat mass of a subject comprising orally administering to the subject a composition produced by the method of any one of claims 8-24.

40. A method of treating, preventing, or reducing inflammation in a subject comprising orally administering to the subject a composition produced by the method of any one of claims 8-24.

41. The method of claim 40, wherein administration of the composition reduces a level of calprotectin in the blood stream or stool of the subject.

42. A method of increasing production of short chain fatty acid (SCFA) in the gut of a subject comprising orally administering to the subject a composition produced by the method of any one of claims 8-24.

43. A method of claim 42, wherein the pH in the gut of the subject is decreased.

44. A method of improving gut barrier integrity in the gut of a subject comprising orally administering to the subject a composition produced by the method of any one of claims 8-24.

45. A method of increasing the growth of commensal bacteria in the gut of a subject comprising orally administering to the subject a composition produced by the method of any one of claims 8-24.

46. The method of claim 45, wherein the commensal bacteria comprise Lactobacillus acidophilus, Lactobacillus reuteri, Akkermansia muciniphila, Bacteroides thetaiotaomicron, Bifidobacterium breve, or Bifidobacterium infantis.

47. The method of claims 26-36, wherein the subject is an infant or toddler.

48. The method of claims 37-46, wherein the subject is an infant or toddler.

49. A method of treating a cancer in a subject in need thereof, comprising administering to the subject the composition of any one of claims 1-7 or the composition produced by the method of any one of claims 8-24.

50. The method of claim 49, wherein the cancer is a solid tumor cancer.

51. The method of claims 49-50, wherein the cancer is an immunotherapy responsive cancer.

52. The method of claims 49-51, wherein the cancer is a checkpoint inhibitor responsive cancer.

53. The method of claim 52, wherein the checkpoint inhibitor is an anti-PD-1 antibody, an anti-CTLA-4 antibody, an anti-PD-L1 antibody, or an anti-PD-L2.

54. The method of claim 52, wherein the checkpoint inhibitor is nivolumab, pembrolizumab, atezolizumab, durvalumab, pidilizumab, PDR001, BMS-936559, avelumab, or SHR-1210.

55. The method of claims 52-54, wherein the checkpoint inhibitor is nivolumab.

56. The method of claims 49-55, wherein the cancer is adrenal cancer, biliary cancer, bladder cancer, brain cancer, breast cancer, cervical cancer, colon cancer, colorectal cancer, rectum cancer, endometrial cancer, esophageal cancer, head or neck cancer, kidney cancer, liver cancer, non-small cell lung cancer, lung cancer, lymphoma, melanoma, meninges cancer, non-melanoma skin cancer, ovarian cancer, pancreatic cancer, prostate cancer, sarcoma, small intestine cancer, or stomach cancer.

57. The method of claims 49-56, wherein the cancer is melanoma or colorectal cancer.

58. The method of claims 52-56, wherein the subject is further administered the checkpoint inhibitor.

59. The method of claim 58, wherein the subject is periodically administered the composition starting at least 1 week, at least 2 weeks, or at least 1 month prior to administration of the checkpoint inhibitor.

60. The method of claim 58, wherein the subject is periodically administered the composition at the same time as the checkpoint inhibitor or starting about 1 week, 2 weeks, or at least 1 month after administration of the checkpoint inhibitor.

61. The method of claims 59-60, wherein periodic administration comprises administration at least once per day, at least once every other day, or at least once every three days.

62. The method of claims 49-61, wherein the composition is orally administered.

63. The method of claims 49-62, wherein administration of the composition increases microbiota diversity in the gut of the subject.

64. The method of claims 49-63, wherein administration of the composition increases infiltration of immune cells into tumors.

65. The method of claim 64, wherein the immune cells are selected from CD4+ T-cells; IFN-γ+, Foxp3+ T-cells; CD8+ T-cells; dendritic cells; plasmacytoid dendritic cells; B cells; macrophages; and natural-killer cells.

66. The method of claims 49-65, wherein administration of the composition reduces systemic inflammation in the subject.

67. The method of claims 49-66, wherein a systemic level of one or more inflammatory cytokines is reduced.

68. The method of claim 67, wherein the inflammatory cytokines are selected from Eotaxin, G-CSF, GM-CSF, IFN-γ, IL-1α, IL-1β, IL-2, IL-3, IL-4, IL-5, IL-6, IL-9, IL-10, IL-12, IL-13, IL-17A, KC, MCP-1 (MCAF), MIP-1α, MIP-1β, RANTES, or TNF-α.

69. The method of claims 49-68, wherein administration of the composition increases cancer cell death.

70. The method of claims 49-69, wherein administration of the composition increases production of short chain fatty acid (SCFA) in the gut of the subject.

71. The method of claims 49-70, wherein administration of the composition reduces the side effects of an anti-cancer therapy administered to the subject.

72. The method of claims 49-69, wherein the subject is human.

73. A method of treating inflammation in a subject in need thereof, comprising administering to the subject the composition of any one of claims 1-7 or the composition produced by the method of any one of claims 8-24.

74. The method of claim 73, wherein the subject has inflammatory bowel disease or acute colitis.

75. The method of claims 73-74, wherein the composition is administered at least once per day, at least once every other day, or at least once every three days.

76. The method of claims 73-75, wherein the composition is orally administered.

77. The method of claims 73-76, wherein administration of the composition increases microbiota diversity in the gut of the subject.

78. The method of claims 73-77, wherein a systemic level of one or more inflammatory cytokines is reduced.

79. The method of claim 78, wherein the inflammatory cytokines are selected from Eotaxin, G-CSF, GM-CSF, IFN-γ, IL-1α, IL-1β, IL-2, IL-3, IL-4, IL-5, IL-6, IL-9, IL-10, IL-12, IL-13, IL-17A, KC, MCP-1 (MCAF), MIP-1α, MIP-1β, RANTES, or TNF-α.

80. The method of claims 73-79, wherein administration of the composition increases production of short chain fatty acid (SCFA) in the gut of the subject.

81. The method of claims 73-80, wherein the subject is human.
